SEQUENCE LISTING (50 sequences)

SEQ ID NO: 1 - DNA, 63 bp, synthetic construct
  source 1..63: mol_type = other DNA
atggccttaccagtgaccgccttgctcctgccgctggccttgctgctccacgccgccaggccg

SEQ ID NO: 2 - protein, 21 aa, synthetic construct
MALPVTALLLPLALLLHAARP

SEQ ID NO: 3 - DNA, 456 bp, synthetic construct
  source 1..456: mol_type = other DNA
gccgtgcagggccccgaggagaccgtgacccaggactgcctgcagctgatcgccgacagcgagacccccaccatccagaagggcagctacaccttcgtgccctggctgctgagcttcaagaggggcagcgccctggaggagaaggagaacaagatcctggtgaaggagaccggctacttcttcatctacggccaggtgctgtacaccgacaagacctacgccatgggccacctgatccagaggaagaaggtgcacgtgttcggcgacgagctgagcctggtgaccctgttcaggtgcatccagaacatgcccgagaccctgcccaacaacagctgctacagcgccggcatcgccaagctggaggagggcgacgagctgcagctggccatccccagggagaacgcccagatcagcctggacggcgacgtgaccttcttcggcgccctgaagctgctg

SEQ ID NO: 4 - protein, 152 aa, synthetic construct [sequence omitted]

SEQ ID NO: 5 - DNA, 21 bp, synthetic construct
  source 1..21: mol_type = other DNA
tctggaggcggctcggatccc

SEQ ID NO: 6 - protein, 7 aa, synthetic construct
SGGGSDP

SEQ ID NO: 7 - DNA, 60 bp, synthetic construct
  source 1..60: mol_type = other DNA
gccgagcccaaatctcctgacaaaactcacacatgcccaccgtgcccaaaagatcccaaa

SEQ ID NO: 8 - protein, 20 aa, synthetic construct
AEPKSPDKTHTCPPCPKDPK

SEQ ID NO: 9 - DNA, 81 bp, synthetic construct
  source 1..81: mol_type = other DNA
ttttgggtgctggtggtggttggtggagtcctggcttgctatagcttgctagtaacagtggcctttattattttctgggtg

SEQ ID NO: 10 - protein, 27 aa, synthetic construct
FWVLVVVGGVLACYSLLVTVAFIIFWV

SEQ ID NO: 11 - DNA, 75 bp, synthetic construct
  source 1..75: mol_type = other DNA
aagacaaacattcgaagctcaacaagagactggaaggaccataaatttaaatggagaaaggaccctcaagacaaa

SEQ ID NO: 12 - protein, 25 aa, synthetic construct
KTNIRSSTRDWKDHKFKWRKDPQDK

SEQ ID NO: 13 - DNA, 360 bp, synthetic construct
  source 1..360: mol_type = other DNA
tggaggagaaagaggaaggagaagcagtcagagaccagtcccaaggaatttttgacaatttacgaagatgtcaaggatctgaaaaccaggagaaatcacgagcaggagcagacttttcctggaggggggagcaccatctactctatgatccagtcccagtcttctgctcccacgtcacaagaacctgcatatacattatattcattaattcagccttccaggaagtctggatccaggaagaggaaccacagcccttccttcaatagcactatctatgaagtgattggaaagagtcaacctaaagcccagaaccctgctcgattgagccgcaaagagctggagaactttgatgtttattcc

SEQ ID NO: 14 - protein, 120 aa, synthetic construct
WRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYS

SEQ ID NO: 15 - DNA, 336 bp, synthetic construct
  source 1..336: mol_type = other DNA
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgcctcctcgc

SEQ ID NO: 16 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 17 - DNA, 486 bp, synthetic construct
  source 1..486: mol_type = other DNA
atgagaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttct

SEQ ID NO: 18 - protein, 162 aa, synthetic construct
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 19 - DNA, 258 bp, synthetic construct
  source 1..258: mol_type = other DNA
gaacggacgatgccccgaattcccaccctgaagaacctagaggatcttgttactgaataccacgggaacttttcggcctggagtggtgtgtctaagggactggctgagagtctgcagccagactacagtgaacgactctgcctcgtcagtgagattcccccaaaaggaggggcccttggggaggggcctggggcctccccatgcaaccagcatagcccctactgggcccccccatgttacaccctaaagcctgaaacc

SEQ ID NO: 20 - protein, 86 aa, synthetic construct
ERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 21 - DNA, 63 bp, synthetic construct
  source 1..63: mol_type = other DNA
gtctctttctgcttggtgatggtactcctttttgcagtggacacaggactatatttctctgtg

SEQ ID NO: 22 - DNA, 63 bp, synthetic construct; identical to SEQ ID NO: 21
  source 1..63: mol_type = other DNA

SEQ ID NO: 23 - DNA, 63 bp, synthetic construct
  source 1..63: mol_type = other DNA
tttttggtgatcatcgtgattctaagcgcactgttccttggcacccttgcctgcttctgtgtg

SEQ ID NO: 24 - protein, 21 aa, synthetic construct
FLVIIVILSALFLGTLACFCV

SEQ ID NO: 25 - DNA, 63 bp, synthetic construct
  source 1..63: mol_type = other DNA
ccattttttttctgctgcttcatcgctgtagccatgggaatccgtttcattattatggtagca

SEQ ID NO: 26 - protein, 21 aa, synthetic construct
PFFFCCFIAVAMGIRFIIMVA

SEQ ID NO: 27 - DNA, 1974 bp, synthetic construct
  source 1..1974: mol_type = other DNA
actagtgccgtgcagggccccgaggagaccgtgacccaggactgcctgcagctgatcgccgacagcgagacccccaccatccagaagggcagctacaccttcgtgccctggctgctgagcttcaagaggggcagcgccctggaggagaaggagaacaagatcctggtgaaggagaccggctacttcttcatctacggccaggtgctgtacaccgacaagacctacgccatgggccacctgatccagaggaagaaggtgcacgtgttcggcgacgagctgagcctggtgaccctgttcaggtgcatccagaacatgcccgagaccctgcccaacaacagctgctacagcgccggcatcgccaagctggaggagggcgacgagctgcagctggccatccccagggagaacgcccagatcagcctggacggcgacgtgaccttcttcggcgccctgaagctgctgtctggaggcggctcggatcccgccgagcccaaatctcctgacaaaactcacacatgcccaccgtgcccaaaagatcccaaattttgggtgctggtggtggttggtggagtcctggcttgctatagcttgctagtaacagtggcctttattattttctgggtgaagacaaacattcgaagctcaacaagagactggaaggaccataaatttaaatggagaaaggaccctcaagacaaatggaggagaaagaggaaggagaagcagtcagagaccagtcccaaggaatttttgacaatttacgaagatgtcaaggatctgaaaaccaggagaaatcacgagcaggagcagacttttcctggaggggggagcaccatctactctatgatccagtcccagtcttctgctcccacgtcacaagaacctgcatatacattatattcattaattcagccttccaggaagtctggatccaggaagaggaaccacagcccttccttcaatagcactatctatgaagtgattggaaagagtcaacctaaagcccagaaccctgctcgattgagccgcaaagagctggagaactttgatgtttattccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgcctcctcgctcgcgaggatctggagcaacaaacttctcactactcaaacaagcaggtgacgtggaggagaatcccgggccttctagacctaggatgagaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttgatacgta

SEQ ID NO: 28 - DNA, 2157 bp, synthetic construct
  source 1..2157: mol_type = other DNA
actagtgccgtgcagggccccgaggagaccgtgacccaggactgcctgcagctgatcgccgacagcgagacccccaccatccagaagggcagctacaccttcgtgccctggctgctgagcttcaagaggggcagcgccctggaggagaaggagaacaagatcctggtgaaggagaccggctacttcttcatctacggccaggtgctgtacaccgacaagacctacgccatgggccacctgatccagaggaagaaggtgcacgtgttcggcgacgagctgagcctggtgaccctgttcaggtgcatccagaacatgcccgagaccctgcccaacaacagctgctacagcgccggcatcgccaagctggaggagggcgacgagctgcagctggccatccccagggagaacgcccagatcagcctggacggcgacgtgaccttcttcggcgccctgaagctgctgtctggaggcggctcggatcccgccgagcccaaatctcctgacaaaactcacacatgcccaccgtgcccaaaagatcccaaattttgggtgctggtggtggttggtggagtcctggcttgctatagcttgctagtaacagtggcctttattattttctgggtggaacggacgatgccccgaattcccaccctgaagaacctagaggatcttgttactgaataccacgggaacttttcggcctggagtggtgtgtctaagggactggctgagagtctgcagccagactacagtgaacgactctgcctcgtcagtgagattcccccaaaaggaggggcccttggggaggggcctggggcctccccatgcaaccagcatagcccctactgggcccccccatgttacaccctaaagcctgaaacctggaggagaaagaggaaggagaagcagtcagagaccagtcccaaggaatttttgacaatttacgaagatgtcaaggatctgaaaaccaggagaaatcacgagcaggagcagacttttcctggaggggggagcaccatctactctatgatccagtcccagtcttctgctcccacgtcacaagaacctgcatatacattatattcattaattcagccttccaggaagtctggatccaggaagaggaaccacagcccttccttcaatagcactatctatgaagtgattggaaagagtcaacctaaagcccagaaccctgctcgattgagccgcaaagagctggagaactttgatgtttattccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgcctcctcgctcgcgaggatctggagcaacaaacttctcactactcaaacaagcaggtgacgtggaggagaatcccgggccttctagacctaggatgagaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttgatacgta

SEQ ID NO: 29 - DNA, 1956 bp, synthetic construct
  source 1..1956: mol_type = other DNA
actagtgccgtgcagggccccgaggagaccgtgacccaggactgcctgcagctgatcgccgacagcgagacccccaccatccagaagggcagctacaccttcgtgccctggctgctgagcttcaagaggggcagcgccctggaggagaaggagaacaagatcctggtgaaggagaccggctacttcttcatctacggccaggtgctgtacaccgacaagacctacgccatgggccacctgatccagaggaagaaggtgcacgtgttcggcgacgagctgagcctggtgaccctgttcaggtgcatccagaacatgcccgagaccctgcccaacaacagctgctacagcgccggcatcgccaagctggaggagggcgacgagctgcagctggccatccccagggagaacgcccagatcagcctggacggcgacgtgaccttcttcggcgccctgaagctgctgtctggaggcggctcggatcccgccgagcccaaatctcctgacaaaactcacacatgcccaccgtgcccaaaagatcccaaagtctctttctgcttggtgatggtactcctttttgcagtggacacaggactatatttctctgtgaagacaaacattcgaagctcaacaagagactggaaggaccataaatttaaatggagaaaggaccctcaagacaaatggaggagaaagaggaaggagaagcagtcagagaccagtcccaaggaatttttgacaatttacgaagatgtcaaggatctgaaaaccaggagaaatcacgagcaggagcagacttttcctggaggggggagcaccatctactctatgatccagtcccagtcttctgctcccacgtcacaagaacctgcatatacattatattcattaattcagccttccaggaagtctggatccaggaagaggaaccacagcccttccttcaatagcactatctatgaagtgattggaaagagtcaacctaaagcccagaaccctgctcgattgagccgcaaagagctggagaactttgatgtttattccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgcctcctcgctcgcgaggatctggagcaacaaacttctcactactcaaacaagcaggtgacgtggaggagaatcccgggccttctagacctaggatgagaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttgatacgta

SEQ ID NO: 30 - DNA, 1956 bp, synthetic construct
  source 1..1956: mol_type = other DNA
actagtgccgtgcagggccccgaggagaccgtgacccaggactgcctgcagctgatcgccgacagcgagacccccaccatccagaagggcagctacaccttcgtgccctggctgctgagcttcaagaggggcagcgccctggaggagaaggagaacaagatcctggtgaaggagaccggctacttcttcatctacggccaggtgctgtacaccgacaagacctacgccatgggccacctgatccagaggaagaaggtgcacgtgttcggcgacgagctgagcctggtgaccctgttcaggtgcatccagaacatgcccgagaccctgcccaacaacagctgctacagcgccggcatcgccaagctggaggagggcgacgagctgcagctggccatccccagggagaacgcccagatcagcctggacggcgacgtgaccttcttcggcgccctgaagctgctgtctggaggcggctcggatcccgccgagcccaaatctcctgacaaaactcacacatgcccaccgtgcccaaaagatcccaaatttttggtgatcatcgtgattctaagcgcactgttccttggcacccttgcctgcttctgtgtgaagacaaacattcgaagctcaacaagagactggaaggaccataaatttaaatggagaaaggaccctcaagacaaatggaggagaaagaggaaggagaagcagtcagagaccagtcccaaggaatttttgacaatttacgaagatgtcaaggatctgaaaaccaggagaaatcacgagcaggagcagacttttcctggaggggggagcaccatctactctatgatccagtcccagtcttctgctcccacgtcacaagaacctgcatatacattatattcattaattcagccttccaggaagtctggatccaggaagaggaaccacagcccttccttcaatagcactatctatgaagtgattggaaagagtcaacctaaagcccagaaccctgctcgattgagccgcaaagagctggagaactttgatgtttattccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgcctcctcgctcgcgaggatctggagcaacaaacttctcactactcaaacaagcaggtgacgtggaggagaatcccgggccttctagacctaggatgagaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttgatacgta

SEQ ID NO: 31 - DNA, 1956 bp, synthetic construct
  source 1..1956: mol_type = other DNA
actagtgccgtgcagggccccgaggagaccgtgacccaggactgcctgcagctgatcgccgacagcgagacccccaccatccagaagggcagctacaccttcgtgccctggctgctgagcttcaagaggggcagcgccctggaggagaaggagaacaagatcctggtgaaggagaccggctacttcttcatctacggccaggtgctgtacaccgacaagacctacgccatgggccacctgatccagaggaagaaggtgcacgtgttcggcgacgagctgagcctggtgaccctgttcaggtgcatccagaacatgcccgagaccctgcccaacaacagctgctacagcgccggcatcgccaagctggaggagggcgacgagctgcagctggccatccccagggagaacgcccagatcagcctggacggcgacgtgaccttcttcggcgccctgaagctgctgtctggaggcggctcggatcccgccgagcccaaatctcctgacaaaactcacacatgcccaccgtgcccaaaagatcccaaaccattttttttctgctgcttcatcgctgtagccatgggaatccgtttcattattatggtagcaaagacaaacattcgaagctcaacaagagactggaaggaccataaatttaaatggagaaaggaccctcaagacaaatggaggagaaagaggaaggagaagcagtcagagaccagtcccaaggaatttttgacaatttacgaagatgtcaaggatctgaaaaccaggagaaatcacgagcaggagcagacttttcctggaggggggagcaccatctactctatgatccagtcccagtcttctgctcccacgtcacaagaacctgcatatacattatattcattaattcagccttccaggaagtctggatccaggaagaggaaccacagcccttccttcaatagcactatctatgaagtgattggaaagagtcaacctaaagcccagaaccctgctcgattgagccgcaaagagctggagaactttgatgtttattccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgcctcctcgctcgcgaggatctggagcaacaaacttctcactactcaaacaagcaggtgacgtggaggagaatcccgggccttctagacctaggatgagaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttgatacgta

SEQ ID NO: 32 - DNA, 123 bp, synthetic construct
  source 1..123: mol_type = other DNA
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 33 - protein, 41 aa, synthetic construct
RSKRSRLLHSDYMNMTPRRPGPTRKHYQPYAPPRDFAAYRS

SEQ ID NO: 34 - DNA, 126 bp, synthetic construct
  source 1..126: mol_type = other DNA
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 35 - protein, 42 aa, synthetic construct
KRGRKKLLYIFKQPFMRPVQTTQEEDGCSCRFPEEEEGGCEL

SEQ ID NO: 36 - DNA, 72 bp, synthetic construct
  source 1..72: mol_type = other DNA
ctgtgcgcacgcccacgccgcagccccgcccaagaagatggcaaagtctacatcaacatgccaggcaggggc

SEQ ID NO: 37 - protein, 24 aa, synthetic construct
LCARPRRSPAQEDGKVYINMPGRG

SEQ ID NO: 38 - DNA, 156 bp, synthetic construct
  source 1..156: mol_type = other DNA
tacttcctgggccggctggtccctcgggggcgaggggctgcggaggcagcgacccggaaacagcgtatcactgagaccgagtcgccttatcaggagctccagggtcagaggtcggatgtctacagcgacctcaacacacagaggccgtattacaaa

SEQ ID NO: 39 - protein, 52 aa, synthetic construct
YFLGRLVPRGRGAAEAATRKQRITETESPYQELQGQRSDVYSDLNTQRPYYK

SEQ ID NO: 40 - DNA, 1554 bp, synthetic construct
  source 1..1554: mol_type = other DNA
actagtgccgtgcagggccccgaggagaccgtgacccaggactgcctgcagctgatcgccgacagcgagacccccaccatccagaagggcagctacaccttcgtgccctggctgctgagcttcaagaggggcagcgccctggaggagaaggagaacaagatcctggtgaaggagaccggctacttcttcatctacggccaggtgctgtacaccgacaagacctacgccatgggccacctgatccagaggaagaaggtgcacgtgttcggcgacgagctgagcctggtgaccctgttcaggtgcatccagaacatgcccgagaccctgcccaacaacagctgctacagcgccggcatcgccaagctggaggagggcgacgagctgcagctggccatccccagggagaacgcccagatcagcctggacggcgacgtgaccttcttcggcgccctgaagctgctgtctggaggcggctcggatcccgccgagcccaaatctcctgacaaaactcacacatgcccaccgtgcccaaaagatcccaaattttgggtgctggtggtggttggtggagtcctggcttgctatagcttgctagtaacagtggcctttattattttctgggtgtggaggagaaagaggaaggagaagcagtcagagaccagtcccaaggaatttttgacaatttacgaagatgtcaaggatctgaaaaccaggagaaatcacgagcaggagcagacttttcctggaggggggagcaccatctactctatgatccagtcccagtcttctgctcccacgtcacaagaacctgcatatacattatattcattaattcagccttccaggaagtctggatccaggaagaggaaccacagcccttccttcaatagcactatctatgaagtgattggaaagagtcaacctaaagcccagaaccctgctcgattgagccgcaaagagctggagaactttgatgtttattccctgtgcgcacgcccacgccgcagccccgcccaagaagatggcaaagtctacatcaacatgccaggcaggggctacttcctgggccggctggtccctcgggggcgaggggctgcggaggcagcgacccggaaacagcgtatcactgagaccgagtcgccttatcaggagctccagggtcagaggtcggatgtctacagcgacctcaacacacagaggccgtattacaaaagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgcctcctcgccatatg

SEQ ID NO: 41 - DNA, 1449 bp, synthetic construct
  source 1..1449: mol_type = other DNA
actagtgccgtgcagggccccgaggagaccgtgacccaggactgcctgcagctgatcgccgacagcgagacccccaccatccagaagggcagctacaccttcgtgccctggctgctgagcttcaagaggggcagcgccctggaggagaaggagaacaagatcctggtgaaggagaccggctacttcttcatctacggccaggtgctgtacaccgacaagacctacgccatgggccacctgatccagaggaagaaggtgcacgtgttcggcgacgagctgagcctggtgaccctgttcaggtgcatccagaacatgcccgagaccctgcccaacaacagctgctacagcgccggcatcgccaagctggaggagggcgacgagctgcagctggccatccccagggagaacgcccagatcagcctggacggcgacgtgaccttcttcggcgccctgaagctgctgtctggaggcggctcggatcccgccgagcccaaatctcctgacaaaactcacacatgcccaccgtgcccaaaagatcccaaattttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcctggaggagaaagaggaaggagaagcagtcagagaccagtcccaaggaatttttgacaatttacgaagatgtcaaggatctgaaaaccaggagaaatcacgagcaggagcagacttttcctggaggggggagcaccatctactctatgatccagtcccagtcttctgctcccacgtcacaagaacctgcatatacattatattcattaattcagccttccaggaagtctggatccaggaagaggaaccacagcccttccttcaatagcactatctatgaagtgattggaaagagtcaacctaaagcccagaaccctgctcgattgagccgcaaagagctggagaactttgatgtttattccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgcctcctcgccatatg

SEQ ID NO: 42 - DNA, 1452 bp, synthetic construct
  source 1..1452: mol_type = other DNA
actagtgccgtgcagggccccgaggagaccgtgacccaggactgcctgcagctgatcgccgacagcgagacccccaccatccagaagggcagctacaccttcgtgccctggctgctgagcttcaagaggggcagcgccctggaggagaaggagaacaagatcctggtgaaggagaccggctacttcttcatctacggccaggtgctgtacaccgacaagacctacgccatgggccacctgatccagaggaagaaggtgcacgtgttcggcgacgagctgagcctggtgaccctgttcaggtgcatccagaacatgcccgagaccctgcccaacaacagctgctacagcgccggcatcgccaagctggaggagggcgacgagctgcagctggccatccccagggagaacgcccagatcagcctggacggcgacgtgaccttcttcggcgccctgaagctgctgtctggaggcggctcggatcccgccgagcccaaatctcctgacaaaactcacacatgcccaccgtgcccaaaagatcccaaattttgggtgctggtggtggttggtggagtcctggcttgctatagcttgctagtaacagtggcctttattattttctgggtgtggaggagaaagaggaaggagaagcagtcagagaccagtcccaaggaatttttgacaatttacgaagatgtcaaggatctgaaaaccaggagaaatcacgagcaggagcagacttttcctggaggggggagcaccatctactctatgatccagtcccagtcttctgctcccacgtcacaagaacctgcatatacattatattcattaattcagccttccaggaagtctggatccaggaagaggaaccacagcccttccttcaatagcactatctatgaagtgattggaaagagtcaacctaaagcccagaaccctgctcgattgagccgcaaagagctggagaactttgatgtttattccaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgcctcctcgccatatg

SEQ ID NO: 43 - DNA, 423 bp, synthetic construct
  source 1..423: mol_type = other DNA
atgcgctggtgtctcctcctgatctgggcccaggggctgaggcaggctcccctcgcctcaggaatgatgacaggcacaatagaaacaacggggaacatttctgcagagaaaggtggctctatcatcttacaatgtcacctctcctccaccacggcacaagtgacccaggtcaactgggagcagcaggaccagcttctggccatttgtaatgctgacttggggtggcacatctccccatccttcaaggatcgagtggccccaggtcccggcctgggcctcaccctccagtcgctgaccgtgaacgatacaggggagtacttctgcatctatcacacctaccctgatgggacgtacactgggagaatcttcctggaggtcctagaaagctcagtggctgagcacggtgccaggttccagattcca

SEQ ID NO: 44 - protein, 141 aa, synthetic construct
MRWCLLLIWAQGLRQAPLASGMMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIP

SEQ ID NO: 45 - DNA, 510 bp, synthetic construct
  source 1..510: mol_type = other DNA
atgcagatcccacaggcgccctggccagtcgtctgggcggtgctacaactgggctggcggccaggatggttcttagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacagagagaagggcagaagtgcccacagcccaccccagcccctcacccaggccagccggccagttccaaaccctggtg

SEQ ID NO: 46 - protein, 170 aa, synthetic construct
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLV

SEQ ID NO: 47 - DNA, 918 bp, synthetic construct
  source 1..918: mol_type = other DNA
ggctggaaccctcacctgctgcttctcctcctgcttgtcatagtcttcattcctgccttctggagcctgaagacccatccattgtggaggctatggaagaagatatgggccgtccccagccctgagcggttcttcatgcccctgtacaagggctgcagcggagacttcaagaaatgggtgggtgcacccttcactggctccagcctggagctgggaccctggagcccagaggtgccctccaccctggaggtgtacagctgccacccaccacggagcccggccaagaggctgcagctcacggagctacaagaaccagcagagctggtggagtctgacggtgtgcccaagcccagcttctggccgacagcccagaactcggggggctcagcttacagtgaggagagggatcggccatacggcctggtgtccattgacacagtgactgtgctagatgcagaggggccatgcacctggccctgcagctgtgaggatgacggctacccagccctggacctggatgctggcctggagcccagcccaggcctagaggacccactcttggatgcagggaccacagtcctgtcctgtggctgtgtctcagctggcagccctgggctaggagggcccctgggaagcctcctggacagactaaagccaccccttgcagatggggaggactgggctgggggactgccctggggtggccggtcacctggaggggtctcagagagtgaggcgggctcacccctggccggcctggatatggacacgtttgacagtggctttgtgggctctgactgcagcagccctgtggagtgtgacttcaccagccccggggacgaaggacccccccggagctacctccgccagtgggtggtcattcctccgccactttcgagccctggaccccaggccagc

SEQ ID NO: 48 - protein, 306 aa, synthetic construct
GWNPHLLLLLLLVIVFIPAFWSLKTHPLWRLWKKIWAVPSPERFFMPLYKGCSGDFKKWVGAPFTGSSLELGPWSPEVPSTLEVYSCHPPRSPAKRLQLTELQEPAELVESDGVPKPSFWPTAQNSGGSAYSEERDRPYGLVSIDTVTVLDAEGPCTWPCSCEDDGYPALDLDAGLEPSPGLEDPLLDAGTTVLSCGCVSAGSPGLGGPLGSLLDRLKPPLADGEDWAGGLPWGGRSPGGVSESEAGSPLAGLDMDTFDSGFVGSDCSSPVECDFTSPGDEGPPRSYLRQWVVIPPPLSSPGPQAS

SEQ ID NO: 49 - DNA, 1359 bp, synthetic construct
  source 1..1359: mol_type = other DNA
actagtgccaccatgcgctggtgtctcctcctgatctgggcccaggggctgaggcaggctcccctcgcctcaggaatgatgacaggcacaatagaaacaacggggaacatttctgcagagaaaggtggctctatcatcttacaatgtcacctctcctccaccacggcacaagtgacccaggtcaactgggagcagcaggaccagcttctggccatttgtaatgctgacttggggtggcacatctccccatccttcaaggatcgagtggccccaggtcccggcctgggcctcaccctccagtcgctgaccgtgaacgatacaggggagtacttctgcatctatcacacctaccctgatgggacgtacactgggagaatcttcctggaggtcctagaaagctcagtggctgagcacggtgccaggttccagattccaggctggaaccctcacctgctgcttctcctcctgcttgtcatagtcttcattcctgccttctggagcctgaagacccatccattgtggaggctatggaagaagatatgggccgtccccagccctgagcggttcttcatgcccctgtacaagggctgcagcggagacttcaagaaatgggtgggtgcacccttcactggctccagcctggagctgggaccctggagcccagaggtgccctccaccctggaggtgtacagctgccacccaccacggagcccggccaagaggctgcagctcacggagctacaagaaccagcagagctggtggagtctgacggtgtgcccaagcccagcttctggccgacagcccagaactcggggggctcagcttacagtgaggagagggatcggccatacggcctggtgtccattgacacagtgactgtgctagatgcagaggggccatgcacctggccctgcagctgtgaggatgacggctacccagccctggacctggatgctggcctggagcccagcccaggcctagaggacccactcttggatgcagggaccacagtcctgtcctgtggctgtgtctcagctggcagccctgggctaggagggcccctgggaagcctcctggacagactaaagccaccccttgcagatggggaggactgggctgggggactgccctggggtggccggtcacctggaggggtctcagagagtgaggcgggctcacccctggccggcctggatatggacacgtttgacagtggctttgtgggctctgactgcagcagccctgtggagtgtgacttcaccagccccggggacgaaggacccccccggagctacctccgccagtgggtggtcattcctccgccactttcgagccctggaccccaggccagccatatg

SEQ ID NO: 50 - DNA, 1446 bp, synthetic construct
  source 1..1446: mol_type = other DNA
actagtgccaccatgcagatcccacaggcgccctggccagtcgtctgggcggtgctacaactgggctggcggccaggatggttcttagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacagagagaagggcagaagtgcccacagcccaccccagcccctcacccaggccagccggccagttccaaaccctggtgggctggaaccctcacctgctgcttctcctcctgcttgtcatagtcttcattcctgccttctggagcctgaagacccatccattgtggaggctatggaagaagatatgggccgtccccagccctgagcggttcttcatgcccctgtacaagggctgcagcggagacttcaagaaatgggtgggtgcacccttcactggctccagcctggagctgggaccctggagcccagaggtgccctccaccctggaggtgtacagctgccacccaccacggagcccggccaagaggctgcagctcacggagctacaagaaccagcagagctggtggagtctgacggtgtgcccaagcccagcttctggccgacagcccagaactcggggggctcagcttacagtgaggagagggatcggccatacggcctggtgtccattgacacagtgactgtgctagatgcagaggggccatgcacctggccctgcagctgtgaggatgacggctacccagccctggacctggatgctggcctggagcccagcccaggcctagaggacccactcttggatgcagggaccacagtcctgtcctgtggctgtgtctcagctggcagccctgggctaggagggcccctgggaagcctcctggacagactaaagccaccccttgcagatggggaggactgggctgggggactgccctggggtggccggtcacctggaggggtctcagagagtgaggcgggctcacccctggccggcctggatatggacacgtttgacagtggctttgtgggctctgactgcagcagccctgtggagtgtgacttcaccagccccggggacgaaggacccccccggagctacctccgccagtgggtggtcattcctccgccactttcgagccctggaccccaggccagccatatg

(19) United States
(12) Patent Application Publication
(10) Pub. No.: US 2026/0216246 A1
(43) Pub. Date: Jul. 30, 2026

(54) CHIMERIC ANTIGEN RECEPTOR CONSTRUCTS WITH OPTIMIZED INTRACELLULAR DOMAINS FOR NATURAL KILLER CELLS

(71) Applicant: CASE WESTERN RESERVE UNIVERSITY, Cleveland, OH (US)

(72) Inventors: Reshmi Parameswaran, Cleveland, OH (US); Derek Wong, Cleveland, OH (US)

(21) Appl. No.: 19/151,841

(22) PCT Filed: Jan. 29, 2024
(86) PCT No.: PCT/US2024/013350

§ 371 Date: Jul. 29, 2025

Related U.S. Application Data
(60) Provisional application No. 63/482,049, filed on Jan. 29, 2023.

(51) Int. Cl. A61K 35/17; A61K 40/15; A61K 40/31; C07K 14/705

(52) U.S. Cl. CPC A61K 35/17; A61K 40/15; A61K 40/31; C07K 14/70575; A61K 2239/17; A61K 2239/21; A61K 2239/22

(57) ABSTRACT

A chimeric antigen receptor (CAR) for NK cells includes an extracellular antigen binding domain, a transmembrane domain, and at least one an intracellular signaling domain.

### RELATED APPLICATION

This application claims priority from U.S. Provisional Application No. 63/482,049, filed Jan. 29, 2023, the subject matter of which is incorporated herein by reference in its entirety.

### SEQUENCE LISTING

The instant application contains a Sequence Listing which has been submitted electronically in XML format and is hereby incorporated by reference in its entirety. Said XML copy, created on Jan. 29, 2024, is named CWR-032309WO ORD st.26 and is 67,733 bytes in size.

### BACKGROUND

Natural Killer Cells (NK cells), are cells of the innate immune system with high antitumor, antiviral and antimicrobial activity. The use of NK cells for the treatment of cancer has attracted interest after successful adoptive transfers and in vivo expansions of NK cells had been reported in patients with cancer (Ruggeri et al (2005) Curr Opin Immunol 17: 211-7; Ren et al (2007) Cancer Biother Radiopharm 22: 223-34; Koehl et al (2004) Blood Cells Mol Dis 33: 261-6.176 Passweg et al (2004) Leukaemia 18:1835-8). In general, donor NK cell infusions were well tolerated without evidence for induction of GvHD in these studies.

A major obstacle of adoptive NK cell infusions in patients with cancer is that only relative small numbers of NK cells can be isolated from regular leukapheresis products. This hampers clinical trials for NK-cell dose dependent anti-tumor responses in humans with cancer. Therefore, ex vivo protocols for expansion and activation of NK cells are under investigation enabling clinical trials at higher NK cell dosages and to permit multiple NK cell infusions. However, most protocols deal with technical disadvantages by using supportive feeder cell lines that could lead to regulatory problems producing NK cell products for large-scale and multi-center trials.

Chimeric Antigen Receptors (CARs) are recombinant receptors that recognize a specific protein or antigen expressed on a target cell. Once expressed in T lymphocytes, which is then called a CAR-T cell, or other cells of the immune system, CARs are able to redirect a specific immune response against all cells that express the antigen they bind to. The most largely explored clinical application of CARs is the cancer immunotherapy, which includes the infusion of cells of the immune system, such as T cells or NK cells, carrying a CAR targeted to a tumor antigen. Such cells are able to generate a strong antitumor response against cells expressing the antigen targeted by the CAR.

The development of NK-cells genetically modified to express a CAR (CAR-NK cells) is still in an earlier stage of development. CAR-NK cells are expected to have several advantages compared to CAR-T cells. In contrast to T cell-based therapies, NK cell-based therapies do not elicit significant levels of cytokine release syndrome or neurotoxicity and are not known to elicit significant graft versus host disease (GvHD). From an efficacy perspective, once engineered with CARs, NK cells should retain their native receptors thus allowing antitumor effect mediated by mechanisms others than those mediated by CAR. However, clinical trials show that CAR-NK therapies tend to exhibit lower efficacy and persistence in the patient.

### SUMMARY

Embodiments described herein relate to chimeric antigen receptor (CAR) constructs with optimized intracellular domains for natural killer (NK) cells. We designed CAR constructs specifically for implementation in NK cells. Their novelty lies in the intracellular signaling domain that follows the transmembrane domain. The constructs utilize novel intracellular signaling domains including an intracellular signaling domain of CD16A, common gamma chain, or γc, 2B4, CD28, 41BB, DAP10, DAP12, and/or combinations thereof. The constructs were found to enhance intracellular signaling elicited by the CAR constructs when stimulated by binding of an extracellular antigen binding domain of the CAR constructs binding to receptors on a target cancer cell. Accordingly, the CAR can include an extracellular antigen binding domain, a transmembrane domain, and at least one intracellular signaling domain wherein the intracellular signaling domain includes a CD16A intracellular signaling domain, a γc intracellular signaling domain, a 2B4 intracellular signaling domain, a CD28 intracellular signaling domain, a 41BB intracellular signaling domain, a DAP10 intracellular signaling domain, a DAP12 intracellular signaling domain, and/or combinations thereof.

In some embodiments the CD16A intracellular signaling domain can include an amino acid sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to SEQ ID NO: 12.

In some embodiments, the ye intracellular signaling domain can include an amino acid sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to SEQ ID NO: 20.

In some embodiments, the 2B4 intracellular signaling domain can include an amino acid sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to SEQ ID NO: 14.

In some embodiments, the CD28 intracellular signaling domain can include an amino acid sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to SEQ ID NO: 33.

In some embodiments, the 41BB intracellular signaling domain can include an amino acid sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to SEQ ID NO: 35.

In some embodiments, the DAP10 intracellular signaling domain can include an amino acid sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to SEQ ID NO: 37.

In some embodiments, the DAP12 intracellular signaling domain can include an amino acid sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to SEQ ID NO: 39.

In some embodiments, the transmembrane domain can include a CD28 transmembrane domain. The CD28 transmembrane domain can include an amino acid sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to SEQ ID NO: 10.

In other embodiments, the transmembrane domain can include a CD16a transmembrane domain. The CD16a transmembrane domain can include an amino acid sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to SEQ ID NO: 22.

In other embodiments, the transmembrane domain can include a 2B4 transmembrane domain. The 2B4 transmembrane domain can include an amino acid sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to SEQ ID NO: 24.

In other embodiments, the transmembrane domain can include a NKG2D transmembrane domain. The NKG2D transmembrane domain can include an amino acid sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to SEQ ID NO: 26.

In other embodiments, the CAR can include a CD3ζ intracellular domain. The CD3ζ intracellular domain can include an amino acid sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to SEQ ID NO: 16.

In some embodiments, the CAR can include at least one of an extracellular spacer or hinge domain The hinge domain can be an IgG1 hinge domain that has, for example, an amino acid sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to SEQ ID NO: 8.

The spacer can include an amino acid sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to SEQ ID NO: 6.

The CAR can further include a signal peptide.

In some embodiments, the CAR from N-to-C-terminus can include an extracellular antigen binding domain, a spacer, an IgG1 hinge domain, a transmembrane domain, a CD16a intracellular signaling domain, a 2B4 intracellular domain, and a CD3ζ intracellular domain. The transmembrane domain can be selected from a CD28 transmembrane domain, a CD16a transmembrane domain, a 2B4 transmembrane domain, or a NKG2D transmembrane domain.

In other embodiments, the CAR from N-to-C-terminus can include an extracellular antigen binding domain, a spacer, an IgG1 hinge domain, a transmembrane domain, a γc intracellular signaling domain, a 2B4 intracellular domain, and a CD3ζ intracellular domain. The transmembrane domain can be selected from a CD28 transmembrane domain, a CD16a transmembrane domain, a 2B4 transmembrane domain, or a NKG2D transmembrane domain.

In other embodiments, the CAR from N-to-C-terminus can include an extracellular antigen binding domain, a spacer, an IgG1 hinge domain, a transmembrane domain, a CD28 intracellular signaling domain, a 2B4 intracellular domain, and a CD3ζ intracellular domain. The transmembrane domain can be selected from a CD28 transmembrane domain, a CD16a transmembrane domain, a 2B4 transmembrane domain, or a NKG2D transmembrane domain.

In other embodiments, the CAR from N-to-C-terminus can include an extracellular antigen binding domain, a spacer, an IgG1 hinge domain, a transmembrane domain, a 2B4 intracellular signaling domain, a 41BB intracellular domain, and a CD3ζ intracellular domain. The transmembrane domain can be selected from a CD28 transmembrane domain, a CD16a transmembrane domain, a 2B4 transmembrane domain, or a NKG2D transmembrane domain.

In other embodiments, the CAR from N-to-C-terminus can include an extracellular antigen binding domain, a spacer, an IgG1 hinge domain, a transmembrane domain, a 2B4 intracellular signaling domain, a DAP10 intracellular domain, a DAP12 intracellular domain, and a CD3ζ intracellular domain. The transmembrane domain can be selected from a CD28 transmembrane domain, a CD16a transmembrane domain, a 2B4 transmembrane domain, or a NKG2D transmembrane domain.

In some embodiments, the extracellular antigen binding domain comprises a polypeptide that binds to a receptor of BAFF. The receptor of BAFF can be selected from the group consisting of B-cell maturation antigen, transmembrane activator and CAML interactor, and BAFF receptor. In one example, the polypeptide can include a BAFF ligand that has, for example, an amino acid sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to SEQ ID NO: 4.

Other embodiments relate to a nucleic acid or nucleotide comprising a nucleotide sequence encoding a CAR described herein. The nucleotide can operably linked to a promoter and provided in an expression construct. The expression construct can include a vector, such as a retroviral vector, a lentiviral vector, or an AAV vector.

In some embodiments, the expression construct can further include a nucleotide sequence encoding a cytokine. The cytokine can include, for example, IL-15, IL-12, IL-2, IL-18, IL-21, or a combination thereof.

In some embodiments, the expression construct can include a nucleotide sequence that encodes the CAR and the cytokine. For example, the nucleotide sequence can be at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to SEQ ID NO: 27, the nucleotide sequence can be at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to SEQ ID NO: 28, the nucleotide sequence can be at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to SEQ ID NO: 29, the nucleotide sequence can be at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to SEQ ID NO: 30, the nucleotide sequence can be at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to SEQ ID NO: 31, the nucleotide sequence can be at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to SEQ ID NO: 40, the nucleotide sequence can be at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to SEQ ID NO: 41, or the nucleotide sequence can be at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to SEQ ID NO: 42.

Other embodiments described herein relate to fusion or chimeric receptor constructs that can be expressed in NK cells to improve NK cell or CAR-NK cell in vivo persistence and efficacy. The fusion receptors can convert immunosuppressive PD-1 or TIGIT signaling into the IL-21 signaling pathway. IL-21 potently reverses NK cell exhaustion and enhances in vivo persistence and antitumor efficacy. Accordingly, the fusion receptor can include a PD-1 or TIGIT extracellular domain, an IL-21R transmembrane domain, and an IL-21R intracellular signaling domain.

In some embodiments, the extracellular domain can include a PD-1 extracellular domain. The PD-1 extracellular domain can include an amino acid sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to SEQ ID NO: 46.

In other embodiments, the extracellular domain can include a TIGIT extracellular domain. The TIGIT extracellular domain can include an amino acid sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to SEQ ID NO: 44.

In some embodiments, the IL-21 transmembrane domain and intracellular domain can include an amino acid sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to SEQ ID NO: 48.

Other embodiments relate to a nucleic acid or nucleotide comprising a nucleotide sequence encoding a fusion receptor described herein. The nucleotide can operably linked to a promoter and provided in an expression construct. The expression construct can include a vector, such as a retroviral vector, a lentiviral vector, or an AAV vector.

In some embodiments, the expression construct encoding the fusion receptor can include a nucleotide sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to SEQ ID NO: 49, or at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to SEQ ID NO: 50.

Still other embodiments relate to an engineered NK cell that includes a CAR and/or a fusion receptor described herein or a natural killer cell transfected or transduced with the expression construct described herein.

In some embodiments, a plurality of the engineered NK cells can be provided in an immunotherapy composition that can be administered to a subject in need thereof.

In some embodiments, the subject can be diagnosed with or have cancer, such as leukemia, lymphoma, myeloma, lung cancer, breast cancer, or head and neck cancer, and the immunotherapy composition can be administered to the subject at a therapeutically amount to treat the cancer in the subject. The immunotherapy composition can optionally be co-administered with one or more chemotherapeutic agents.

In other embodiments, the subject can have an autoimmune disease, such as systemic lupus erythematosus, Sjorgen's syndrome, narcolepsy, diabetes, pancreatitis, Crohn's disease, celiac disease, ankylosing spondylitis, psoriasis, Grave's disease, or rheumatoid arthritis, and the immunotherapy composition can be administered to the subject at a therapeutically amount to treat the autoimmune disease in the subject.

In some embodiments, the immunotherapy composition administered to the subject can include at least about 1 million engineered CAR-NK cells, at least about 2 million engineered CAR-NK cells, at least about 3 million engineered CAR-NK cells, at least about 4 million engineered CAR-NK cells, at least about 5 million engineered CAR-NK cells, or at least about 10 million engineered CAR-NK cells.

### BRIEF DESCRIPTION OF THE DRAWINGS

FIG. 1 illustrates a diagram of previous reported BAFF-CAR construct and novel CAR-NK constructs. Construct 1: previously reported BAFF-CAR construct; Construct 2: novel CAR-NK construct utilizing CD16a intracellular signaling domain; Construct 3: novel CAR-NK construct utilizing γc intracellular signaling domain.

FIG. 2 illustrates a diagram of lentiviral expression vectors used to produce CAR construct lentivirus. The transgene coding for the CAR construct is placed downstream of the SFFV promoter. The CAR transgene is expressed in tandem with a puromycin resistance gene (PuroR) to allow for puromycin-mediated selection of successfully transduced NK cells using puromycin. In the pHR-CAR-IL-15-PuroR plasmid, the IL-15 transgene is inserted between the P2A and T2A self-cleaving peptide sequences to permit simultaneous expression of CAR, IL-15, and PuroR transgenes by the CAR-NK cell.

FIG. 3 illustrates validation of BAFF surface expression on CAR-NK cells. NK92 cells transduced with different BAFF-CAR constructs were stained with anti-BAFF antibody and analyzed via flow cytometry to confirm transduction and surface expression of BAFF-CAR construct. Cells transduced with the regular vector or the IL-15 coding vector were analyzed separately.

FIG. 4 illustrates validation of IL-15 secretion from BAFF CAR-NK cells. 1e5 NK92 cells expressing either empty vector control, the −CD28-OX40-CD3ζ construct, or the −CD16A-2B4-CD3ζ construct through the pHR-CAR-IL-15-PuroR vector were seeded in a 24-well plate with 1 mL media supplemented with 200 U/mL IL-2. Supernatant was collected 72 h later and analyzed for IL-15 secretion via ELISA.

FIG. 5 illustrates BAFF CAR-NK cells with the −CD16A-2B4-CD3ζ design exhibit superior cytotoxicity. BAFF CAR-NK cells expressing different constructs were co-cultured with fluorescently-labeled Jeko-1 cells at different effector:target ratios for 16 h. Cells were stained with propidium iodide (PI), and flow cytometry was used to gate on labeled cells and measure cancer cell death. ns=not significant, **P<0.01, ***P<0.001, ****P<0.0001. Graphs display mean+/−SD, n=3 biological co-cultures, 2-way ANOVA with Dunnett's multiple comparisons test. All samples are being compared to the original CAR construct (blue).

FIGS. 6(A-C) illustrate BAFF CAR-NK cells exhibit significant cytotoxicity while secreting much lower levels of pro-inflammatory cytokines compared to BAFF CAR-T cells. A) The BAFF-CAR construct was expressed in primary human T cells and primary human NK cells via lentiviral transduction. Lentiviral transduction efficiency was measured, and the percentage of CAR-T cells in the T cell population was matched to that of CAR-NK cells in the NK cell population by diluting with untransduced T cells. % efficiency was determined based on % GFP expression and correlated exogenous surface expression of BAFF. CD3 and CD56 were measured in the T cell and NK cell populations to confirm cell type purity. B) Untransduced T cells, CAR-T cells, untransduced NK cells, or CAR-NK cells were co-cultured with fluorescently-labeled Jeko-1 MCL cells at 2:1 effector:target (E:T) ratio for 16 h, followed by flow cytometry. Cytotoxicity was measured via propidium iodide (PI) staining and gating on labeled target cells. ****P<0.0001. Graphs display mean+/−SD, n=3 biologically independent co-cultures, 1-way ANOVA with Sfik's multiple comparisons test. C) Co-culture supernatant was collected from cytotoxicity assays to measure T cell and NK cell release of various pro-inflammatory cytokines and lytic enzymes using a multiplex cytokine release assay. *P<0.05. **P<0.01, ***P<0.001, ****P<0.0001. Mean±SD, n=3 biologically independent co-culture samples, 1-way ANOVA with Tukey's multiple comparisons test.

FIGS. 7(A-C) illustrate NK92 cells were transduced with novel BAFF CAR-NK constructs and compared with the original BAFF-CAR construct. A) Schematics of novel BAFF CAR-NK constructs, which consist of extracellular BAFF ligand and different intracellular signaling domains. B) BAFF-CAR surface expression was measured in NK92 cells transduced with each of the BAFF CAR-NK constructs using flow cytometry. Untransduced NK92 cells serve as a negative control. MFI=median fluorescence intensity. C) BAFF CAR-NK92 cells were co-cultured with luciferase-expressing Jeko-1 cells at various effector:target (E:T) ratios for 16 h, followed by flow cytometry. Cytotoxicity was measured via propidium iodide (PI) staining and gating on labeled target cells. Ns=not significant, **P<0.01, ***P<0.001, ****P<0.0001. Graphs display mean+/−SD, n=3 biologically independent co-cultures, 2-way ANOVA with Dunnett's multiple comparisons test.

FIGS. 8(A-H) illustrate BAFF CAR-NK cells can be engineered to secrete IL-15, which display persistence in different organ systems in vivo using an intravenous MCL xenograft model. A) Map of the custom Phr lentiviral vector coding simultaneously for BAFF-CAR expression and IL-15 secretion. The human IL-15 transgene was inserted between the P2A and T2A self-cleaving peptide sequences. B) NK92 cells transduced with the baff-CAR construct coding for IL-15 with the original IL-15 signal peptide (WT IL-15) or the IL-2 signal peptide (IL-2 Signal Peptide IL-15) were plated in a 24-well plate (1e5 cells in 1 Ml complete media) for 48 h or 72 h. Cell culture supernatant was collected, and IL-15 concentration was measured via ELISA. Untransduced NK92 cells serve as negative control. ****P<0.0001. Graphs display mean+/−SD, n=3 biologically independent cultures, 2-way ANOVA with Dunnett's multiple comparisons test. C) NK92 cells transduced with the baff-CAR construct coding for IL-15 with the original IL-15 signal peptide (CAR-IL-15) and untransduced NK92 cells were plated in a 24-well plate (1e5 cells in 1 Ml media without recombinant IL-2). Cell count was measured using the Countess 3 automated cell counter multiple days after seeding. Cells were split into new culture vessels as they became confluent. **P<0.01. Graphs display mean+/−SD, n=3 biologically independent samples, multiple unpaired two-tailed t-tests with Holm-Šidák correction for multiple comparisons. D) Schematic for i.v. MCL xenograft model. NSG mice were injected i.v. via tail vein with 1e6 Jeko-1-luc cells on Day 0. BAFF CAR-NK92 cells expressing IL-15 using the original IL-15 signal peptide (WT IL-15 CAR) or the IL-2 signal peptide (IL-2 SP IL-15 CAR), untransduced NK92 cells, or PBS were injected i.v. at Days 5, 8, and 12 post-tumor inoculation. E) Bioluminescence imaging was performed weekly up to Day 31 post-tumor inoculation. N=5 mice per treatment group. F) Kaplan-Meier survival curves were generated, with Day 31 post-inoculation serving as the endpoint of the experiment. G) Average radiance or total flux of mice from bioluminescence imaging are plotted over time up to Day 27 post-inoculation. Ns=not significant, ****P<0.0001. Graphs display mean+/−SD, n=5 mice, 2-way ANOVA with Dunnett's multiple comparisons test. H) Spleen, bone marrow, cervical lymph nodes, and liver were collected from mice at time of euthanasia to measure presence of NK92 cells using flow cytometry. Single cell suspensions were obtained, followed by staining for human BAFF and CD56. Box within flow dot plot indicates human CD56+ population.

FIGS. 9(A-B) illustrate BAFF CAR-NK cells exhibit significant cytotoxicity against multiple MCL and MM cell lines in vitro. A) 10 BAFF CAR-NK or untransduced NK cells were co-cultured with fluorescently-labeled Jeko-1, JVM2, and MM.1s cells at various E:T ratios for 16 h, followed by flow cytometry. Cytotoxicity was measured via propidium iodide (PI) staining and gating on labeled target cells. ***P<0.001, **** P<0.0001. Graphs display mean+/−SD, n=3 biologically independent co-cultures, multiple unpaired two-tailed t-tests with Holm-Šidák correction for multiple comparisons. B) NK cells were co-cultured with Jeko-1, JVM2, and MM.1s cells or no cancer cells at 2.5:1 E:T ratio for 6 h while staining for the degranulation marker CD107a. % CD107a+ NK cells were measured via flow cytometry after gating on CD56+ cells; for CAR-NK samples, additional gating over GFP+ cells was applied to exclude unmodified cells. ***P<0.001, **** P<0.0001. Graphs display mean+/−SD, n=3 biologically independent co-cultures, multiple unpaired two-tailed t-tests with Holm-Šidák correction for multiple comparisons.

FIGS. 10(A-D) illustrate higher doses of 1° BAFF CAR-NK cells may be required to better demonstrate efficacy in an intravenous MCL xenograft model. A) NSG mice were injected i.v. via tail vein with 1e6 Jeko-1-luc cells on Day 0.1° BAFF CAR-NK cells expressing the CD28-OX40 or CD16A-2B4 construct, untransduced NK cells (Control NK), or PBS were injected i.v. at Days 2, 9, and 16 post-tumor inoculation. Bioluminescence imaging was performed weekly up to Day 28 post-tumor inoculation. N=5 mice per treatment group except n=4 mice in CD28-OX40 BAFF CAR-NK group. B) Average radiance of the mice in each treatment group are displayed for Days 21 and 28 post-tumor inoculation. Ns=not significant, *P<0.05. Graphs display mean+/−SD, n=5 mice per treatment group except n=4 mice in CD28 BAFF CAR-NK group, 1-way ANOVA with Dunnett's multiple comparisons test. C) On Day 23 post-tumor inoculation, mouse blood was drawn via tail vein from mice treated with CD28-OX40 or CD16A-2B4 BAFF CAR-NK cells or untransduced NK cells (Ctrl). Samples were processed, stained for human CD56, mixed with counting beads, and analyzed via flow cytometry to determine human NK cell count in mouse peripheral blood. D) BAFF CAR-NK cells were produced using an improved NK cell transduction and expansion protocol optimized during this project year, achieving both higher transduction efficiency and cell quantity.

FIG. 11 illustrates the schematics of the proposed PD-1/IL-21 and TIGIT/IL-21 fusion receptors. These fusion receptors combine either the PD-1 extracellular or TIGIT extracellular domain with the transmembrane and intracellular domains of the IL-21 receptor.

### DETAILED DESCRIPTION

Methods involving conventional molecular biology techniques are described herein. Such techniques are generally known in the art and are described in detail in methodology treatises, such as Current Protocols in Molecular Biology, ed. Ausubel et al., Greene Publishing and Wiley-Interscience, New York, 1992 (with periodic updates). Unless otherwise defined, all technical terms used herein have the same meaning as commonly understood by one of ordinary skill in the art to which the present invention pertains. Commonly understood definitions of molecular biology terms can be found in, for example, Rieger et al., Glossary of Genetics: Classical and Molecular, 5^{th} Ed., Springer-Verlag: New York, 1991, and Lewin, Genes V, Oxford University Press: New York, 1994. The definitions provided herein are to facilitate understanding of certain terms used frequently herein and are not meant to limit the scope of the present invention.

It must be noted that, as used in the specification and the appended claims, the singular forms “a,” “an” and “the” include plural referents unless the context clearly dictates otherwise. Thus, for example, reference to “a pharmaceutical carrier” includes mixtures of two or more such carriers, and the like. “Optional” or “optionally” means that the subsequently described event or circumstance can or cannot occur, and that the description includes instances where the event or circumstance occurs and instances where it does not.

As used herein, the term “about” means that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art. When the term “about” is used in describing a value or an endpoint of a range, the disclosure should be understood to include the specific value or endpoint referred to. Whether or not a numerical value or endpoint of a range in the specification recites “about,” the numerical value or endpoint of a range is intended to include two embodiments: one modified by “about,” and one not modified by “about.” It will be further understood that the endpoints of each of the ranges are significant both in relation to the other endpoint, and independently of the other endpoint.

The term “about” or “approximately” refers to a quantity, level, value, number, frequency, percentage, dimension, size, amount, weight or length that varies by as much as 15%, 10%, 9%, 8%, 7%, 6%, 5%, 4%, 3%, 2% or 1% to a reference quantity, level, value, number, frequency, percentage, dimension, size, amount, weight or length. In one embodiment, the term “about” or “approximately” refers a range of quantity, level, value, number, frequency, percentage, dimension, size, amount, weight or length±15%, ±10%, ±9%, ±8%, ±7%, ±6%, ±5%, ±4%, ±3%, ±2%, or ±1% about a reference quantity, level, value, number, frequency, percentage, dimension, size, amount, weight or length.

The terms “substantial,” “substantially,” and variations thereof as used herein are intended to note that a described feature is equal or approximately equal to a value or description. For example, a “substantially planar” surface is intended to denote a surface that is planar or approximately planar. Moreover, “substantially” is intended to denote that two values are equal or approximately equal. In some embodiments, “substantially” may denote values within about 10% of each other, such as within about 5% of each other, or within about 2% of each other.

It is noted that the terms “substantially” and “about” may be utilized herein to represent the inherent degree of uncertainty that may be attributed to any quantitative comparison, value, measurement, or other representation. These terms are also utilized herein to represent the degree by which a quantitative representation may vary from a stated reference without resulting in a change in the basic function of the subject matter at issue. Thus, cells that are “free of” or “substantially free of T cell contamination” for example, are cells to which T cells are not actively added or batched into cell culture, but may be present in very small as a contaminant resulting from natural cell progression during expansion. Similarly, other components may be characterized as “free of” or “substantially free of” in the same manner. Further, as used herein, the term “consisting essentially of” allows for elements not explicitly recited but excludes element that affect basic or novel characteristics of the inventions. As recited herein, the term “consisting of” excludes elements not expressly stated.

The term “engineered” as used herein refers to an entity that is generated by the hand of man, including a cell, nucleic acid, polypeptide, vector, and so forth. In at least some cases, an engineered entity is synthetic and comprises elements that are not naturally present or configured in the manner in which it is utilized in the disclosure. In specific embodiments, a vector is engineered through recombinant nucleic acid technologies, and a cell is engineered through transfection or transduction of an engineered vector.

The term “Nucleic acids,” “nucleic acid molecules,” “nucleotides,” “nucleotide(s) sequence,” and “polynucleotide” are used interchangeably and refer to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogs thereof, such as phosphorothioates and thioesters, in either single stranded form, or a double-stranded helix. Single stranded nucleic acid sequences refer to single-stranded DNA (ssDNA) or single-stranded RNA (ssRNA). Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear or circular DNA molecules (e.g., restriction fragments), plasmids, supercoiled DNA and chromosomes. In discussing the structure of particular double-stranded DNA molecules, sequences can be described herein according to the normal convention of giving only the sequence in the 5′ to 3′ direction along the non-transcribed strand of DNA (i.e., the strand having a sequence homologous to the Mrna). A “recombinant DNA molecule” is a DNA molecule that has undergone a molecular biological manipulation. DNA includes, but is not limited to, Cdna, genomic DNA, plasmid DNA, synthetic DNA, and semi-synthetic DNA. A “nucleic acid composition” of the disclosure comprises one or more nucleic acids as described herein.

As used herein, a “coding region” or “coding sequence” is a portion of polynucleotide which consists of codons translatable into amino acids. Although a “stop codon” (TAG, TGA, or TAA) is typically not translated into an amino acid, it can be considered to be part of a coding region, but any flanking sequences, for example promoters, ribosome binding sites, transcriptional terminators, introns, and the like, are not part of a coding region. The boundaries of a coding region are typically determined by a start codon at the 5′ terminus, encoding the amino terminus of the resultant polypeptide, and a translation stop codon at the 3′ terminus, encoding the carboxyl terminus of the resulting polypeptide. Two or more coding regions can be present in a single polynucleotide construct, e.g., on a single vector, or in separate polynucleotide constructs, e.g., on separate (different) vectors. It follows, then, that a single vector can contain just a single coding region, or comprise two or more coding regions.

The term “downstream” refers to a nucleotide sequence that is located 3′ to a reference nucleotide sequence. In certain embodiments, downstream nucleotide sequences relate to sequences that follow the starting point of transcription. For example, the translation initiation codon of a gene is located downstream of the start site of transcription.

The term “upstream” refers to a nucleotide sequence that is located 5′ to a reference nucleotide sequence. In certain embodiments, upstream nucleotide sequences relate to sequences that are located on the 5′ side of a coding region or starting point of transcription. For example, most promoters are located upstream of the start site of transcription.

The term “expression” as used herein refers to a process by which a polynucleotide produces a gene product, for example, an RNA or a polypeptide. It includes without limitation transcription of the polynucleotide into messenger RNA (Mrna), transfer RNA (Trna), small hairpin RNA (shRNA), small interfering RNA (siRNA) or any other RNA product, and the translation of an Mrna into a polypeptide. Expression produces a “gene product.” As used herein, a gene product can be either a nucleic acid, e.g., a messenger RNA produced by transcription of a gene, or a polypeptide which is translated from a transcript. Gene products described herein further include nucleic acids with post transcriptional modifications, e.g., polyadenylation or splicing, or polypeptides with post translational modifications, e.g., methylation, glycosylation, the addition of lipids, association with other protein subunits, or proteolytic cleavage. The term “yield,” as used herein, refers to the amount of a polypeptide produced by the expression of a gene.

A “vector” refers to any vehicle for the cloning of and/or transfer of a nucleic acid into a host cell. A vector can be a replicon to which another nucleic acid segment can be attached so as to bring about the replication of the attached segment. A “replicon” refers to any genetic element (e.g., plasmid, phage, cosmid, chromosome, virus) that functions as an autonomous unit of replication in vivo, i.e., capable of replication under its own control. The term “vector” includes vehicles for introducing the nucleic acid into a cell in vitro, ex vivo or in vivo. A large number of vectors are known and used in the art including, for example, plasmids, modified eukaryotic viruses, or modified bacterial viruses. Insertion of a polynucleotide into a suitable vector can be accomplished by ligating the appropriate polynucleotide fragments into a chosen vector that has complementary cohesive termini.

Vectors can be engineered to encode selectable markers or reporters that provide for the selection or identification of cells that have incorporated the vector. Expression of selectable markers or reporters allows identification and/or selection of host cells that incorporate and express other coding regions contained on the vector. Examples of selectable marker genes known and used in the art include: genes providing resistance to ampicillin, streptomycin, gentamycin, kanamycin, hygromycin, sulfonamide, puromycin, and the like; and genes that are used as phenotypic markers, i.e., anthocyanin regulatory genes, sopentenyl transferase gene, and the like. Examples of reporters known and used in the art include: luciferase (Luc), green fluorescent protein (GFP), chloramphenicol acetyltransferase (CAT), β-galactosidase (LacZ), β-glucuronidase (Gus), and the like. Selectable markers can also be considered to be reporters.

The term “heterologous” means derived from a genotypically distinct entity from that of the rest of the entity to which it is compared or into which it is introduced or incorporated. For example, a polynucleotide introduced by genetic engineering techniques into a different cell type is a heterologous polynucleotide (and, when expressed, can encode a heterologous polypeptide). Similarly, a cellular sequence (e.g., a gene or portion thereof) that is incorporated into a viral vector is a heterologous nucleotide sequence with respect to the vector.

The term “heterologous gene” or “heterologous nucleic acid” refers to a gene that does not naturally occur as part of a viral genome. For instance, a heterologous gene can be a mammalian gene, e.g., a therapeutic gene, e.g., a mammalian gene that encodes a therapeutic protein. In some embodiments, a heterologous gene encodes a protein or portion thereof that is defective or absent in the target cell and/or subject. In some embodiments, the heterologous gene contains one or more exons encoding a protein that is defective or absent in the target cell and/or subject. For example, in some embodiments, the heterologous gene includes one or more trans-splicing molecules, e.g., as described in WO 2017/087900, which is incorporated herein by reference in its entirety. In some embodiments, a heterologous gene includes a therapeutic nucleic acid, such as a therapeutic RNA (e.g., microRNA).

The term “promoter” refers to a sequence that regulates transcription of a heterologous gene operably linked to the promoter. Promoters provide the sequence sufficient to direct transcription and/or recognition sites for RNA polymerase and other transcription factors required for efficient transcription and can direct cell-specific expression. In addition to the sequence sufficient to direct transcription, a promoter sequence of the invention can also include sequences of other regulatory elements that are involved in modulating transcription (e.g., enhancers, kozak sequences, and introns).

The term “target cell” refers to any cell that expresses a target gene and which the vector infects or is intended to infect. Vectors can infect target cells that reside in a subject (in situ) or target cells in culture.

The term “host cell” as used herein refers to, for example microorganisms, yeast cells, insect cells, and mammalian cells, that can be, or have been, used as recipients of ssDNA or vectors. The term includes the progeny of the original cell which has been transduced. Thus, a “host cell” as used herein generally refers to a cell which has been transduced with an exogenous DNA sequence. It is understood that the progeny of a single parental cell may not necessarily be completely identical in morphology or in genomic or total DNA complement to the original parent, due to natural, accidental, or deliberate mutation. In some embodiments, the host cell can be an in vitro host cell.

The term “subject” generally refers to an individual having a biological sample that is undergoing processing or analysis and, in specific cases, has or is suspected of having cancer. The subject can be any organism or animal subject that is an object of a method or material, including mammals, e.g., humans, laboratory animals (e.g., primates, rats, mice, rabbits), livestock (e.g., cows, sheep, goats, pigs, turkeys, and chickens), household pets (e.g., dogs, cats, and rodents), horses, and transgenic non-human animals. The subject can be a patient, e.g., have or be suspected of having a disease (that may be referred to as a medical condition), such as benign or malignant neoplasia, or cancer. The subject may be undergoing or having undergone treatment. The subject may be asymptomatic. The subject may be healthy individuals but that are desirous of prevention of cancer. The term “individual” may be used interchangeably, in at least some cases. The “subject” or “individual”, as used herein, may or may not be housed in a medical facility and may be treated as an outpatient of a medical facility. The individual may be receiving one or more medical compositions via the internet. An individual may comprise any age of a human or non-human animal and therefore includes both adult and juveniles (i.e., children) and infants and includes in utero individuals. It is not intended that the term connote a need for medical treatment, therefore, an individual may voluntarily or involuntarily be part of experimentation whether clinical or in support of basic science studies.

The terms “treatment” or “treating,” includes any beneficial or desirable effect on the symptoms or pathology of a disease or pathological condition, and may include even minimal reductions in one or more measurable markers of the disease or condition being treated, e.g., cancer. Treatment can involve optionally either the reduction or amelioration of symptoms of the disease or condition, or the delaying of the progression of the disease or condition. “Treatment” does not necessarily indicate complete eradication or cure of the disease or condition, or associated symptoms thereof.

The term cancer refers to all types of cancer, neoplasm or malignant tumors found in mammals, including leukemias, lymphomas, melanomas, neuroendocrine tumors, carcinomas and sarcomas. Exemplary cancers that may be treated with a compound, pharmaceutical composition, or method provided herein include lymphoma, sarcoma, bladder cancer, bone cancer, brain tumor, cervical cancer, colon cancer, esophageal cancer, gastric cancer, head and neck cancer, kidney cancer, myeloma, thyroid cancer, leukemia, prostate cancer, breast cancer (e.g., triple negative, ER positive, ER negative, chemotherapy resistant, asophili resistant, HER2 positive, doxorubicin resistant, tamoxifen resistant, ductal carcinoma, lobular carcinoma, primary, metastatic), ovarian cancer, pancreatic cancer, liver cancer (e.g., hepatocellular carcinoma), lung cancer (e.g., non-small cell lung carcinoma, squamous cell lung carcinoma, adenocarcinoma, large cell lung carcinoma, small cell lung carcinoma, carcinoid, sarcoma), glioblastoma multiforme, glioma, melanoma, prostate cancer, castration-resistant prostate cancer, breast cancer, triple negative breast cancer, glioblastoma, ovarian cancer, lung cancer, squamous cell carcinoma (e.g., head, neck, or esophagus), colorectal cancer, leukemia, acute myeloid leukemia, lymphoma, B cell lymphoma, or multiple myeloma. Additional examples include, cancer of the thyroid, endocrine system, brain, breast, cervix, colon, head & neck, esophagus, liver, kidney, lung, non-small cell lung, melanoma, mesothelioma, ovary, sarcoma, stomach, uterus or Medulloblastoma, Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic asophilic, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, Paget's Disease of the Nipple, Phyllodes Tumors, Lobular Carcinoma, Ductal Carcinoma, cancer of the pancreatic stellate cells, cancer of the hepatic stellate cells, or prostate cancer.

The term leukemia refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound, pharmaceutical composition, or method provided herein include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, asophilic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

The terms metastasis, and metastatic cancer can be used interchangeably and refer to the spread of a proliferative disease or disorder, e.g., cancer, from one organ or another non-adjacent organ or body part. Cancer occurs at an originating site, e.g., breast, which site is referred to as a primary tumor, e.g., primary breast cancer. Some cancer cells in the primary tumor or originating site acquire the ability to penetrate and infiltrate surrounding normal tissue in the local area and/or the ability to penetrate the walls of the lymphatic system or vascular system circulating through the system to other sites and tissues in the body. A second clinically detectable tumor formed from cancer cells of a primary tumor is referred to as a metastatic or secondary tumor. When cancer cells metastasize, the metastatic tumor and its cells are presumed to be similar to those of the original tumor. Thus, if lung cancer metastasizes to the breast, the secondary tumor at the site of the breast consists of abnormal lung cells and not abnormal breast cells. The secondary tumor in the breast is referred to a metastatic lung cancer. Thus, the phrase metastatic cancer refers to a disease in which a subject has or had a primary tumor and has one or more secondary tumors. The phrases non-metastatic cancer or subjects with cancer that is not metastatic refers to diseases in which subjects have a primary tumor but not one or more secondary tumors. For example, metastatic lung cancer refers to a disease in a subject with or with a history of a primary lung tumor and with one or more secondary tumors at a second location or multiple locations, e.g., in the breast.

The term associated or associated with in the context of a substance or substance activity or function associated with a disease (e.g., cancer (e.g., leukemia, lymphoma, B cell lymphoma, or multiple myeloma)) means that the disease (e.g., cancer, (e.g., leukemia, lymphoma, B cell lymphoma, or multiple myeloma)) is caused by (in whole or in part), or a symptom of the disease is caused by (in whole or in part) the substance or substance activity or function.

An autoimmune disease refers to a disease or disorder that arises from altered immune reactions by the immune system of a subject, e.g., against substances tissues and/or cells normally present in the body of the subject. Autoimmune diseases include, but are not limited to, arthritis, rheumatoid arthritis, psoriatic arthritis, juvenile idiopathic arthritis, scleroderma, systemic scleroderma, multiple sclerosis, systemic lupus erythematosus (SLE), myasthenia gravis, juvenile onset diabetes, diabetes mellitus type 1, Guillain-Barre syndrome, Hashimoto's encephalitis, Hashimoto's thyroiditis, ankylosing spondylitis, psoriasis, Sjogren's syndrome, vasculitis, glomerulonephritis, auto-immune thyroiditis, Behcet's disease, Crohn's disease, ulcerative colitis, bullous pemphigoid, sarcoidosis, psoriasis, ichthyosis, Graves ophthalmopathy, inflammatory bowel disease, Addison's disease, Vitiligo, asthma, and allergic asthma.

All percentages and ratios used herein, unless otherwise indicated, are by weight.

Embodiments described herein relate to chimeric antigen receptor (CAR) constructs with optimized intracellular domains for natural killer (NK) cells, nucleic acids encoding the CARs, expression constructs that include the nucleic acids for transducing or transfecting NK cells, engineered NK cells transfected or transduced with the expression constructs, immunotherapy compositions including the engineered NK cells, and methods of treating cancer or an autoimmune disease with the immunotherapy composition.

We designed CAR constructs specifically for implementation in NK cells. Their novelty lies in the intracellular signaling domain that follows the transmembrane domain. The constructs utilize novel intracellular signaling domains including an intracellular signaling domain of CD16A, common gamma chain, or γc, 2B4, CD28, 41BB, DAP10, DAP12, and/or combinations thereof. The constructs were found to enhance intracellular signaling elicited by the CAR constructs when stimulated by binding of an extracellular antigen binding domain of the CAR constructs binding to receptors on a target cancer cell. Accordingly, the CAR can include an extracellular antigen binding domain, a transmembrane domain, and at least one intracellular signaling domain wherein the intracellular signaling domain includes a CD16A intracellular signaling domain, a γc intracellular signaling domain, a 2B4 intracellular signaling domain, a CD28 intracellular signaling domain, a 41BB intracellular signaling domain, a DAP10 intracellular signaling domain, a DAP12 intracellular signaling domain, and/or combinations thereof.

In some embodiments the CD16A intracellular signaling domain can include an amino acid sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to SEQ ID NO: 12.

In some embodiments, the ye intracellular signaling domain can include an amino acid sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to SEQ ID NO: 20.

In some embodiments, the 2B4 intracellular signaling domain can include an amino acid sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to SEQ ID NO: 14.

In some embodiments, the CD28 intracellular signaling domain can include an amino acid sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to SEQ ID NO: 33.

In some embodiments, the 41BB intracellular signaling domain can include an amino acid sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to SEQ ID NO: 35.

In some embodiments, the DAP10 intracellular signaling domain can include an amino acid sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to SEQ ID NO: 37.

In some embodiments, the DAP12 intracellular signaling domain can include an amino acid sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to SEQ ID NO: 39.

In other embodiments, the CAR can further include a CD3ζ intracellular domain. The CD3ζ intracellular domain can include an amino acid sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to SEQ ID NO: 16.

In some embodiments, the transmembrane domain can include a CD28 transmembrane domain. The CD28 transmembrane domain can include an amino acid sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to SEQ ID NO: 10.

In other embodiments, the transmembrane domain can include a CD16a transmembrane domain. The CD16a transmembrane domain can include an amino acid sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to SEQ ID NO: 22.

In other embodiments, the transmembrane domain can include a 2B4 transmembrane domain. The 2B4 transmembrane domain can include an amino acid sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to SEQ ID NO: 24.

In other embodiments, the transmembrane domain can include a NKG2D transmembrane domain. The NKG2D transmembrane domain can include an amino acid sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to SEQ ID NO: 26.

In some embodiments, the CAR can include at least one of an extracellular spacer or hinge domain. The hinge domain can be an IgG1 hinge domain that has, for example, an amino acid sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to SEQ ID NO: 8.

The spacer can include an amino acid sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to SEQ ID NO: 6.

The CAR can further include a signal peptide.

In some embodiments, the CAR from N-to-C terminus can include an extracellular antigen binding domain, a spacer, an IgG1 hinge domain, a transmembrane domain, a CD16a intracellular signaling domain, a 2B4 intracellular domain, and a CD3ζ intracellular domain. The transmembrane domain can be selected from a CD28 transmembrane domain, a CD16a transmembrane domain, a 2B4 transmembrane domain, or a NKG2D transmembrane domain.

In other embodiments, the CAR can include an extracellular antigen binding domain, a spacer, an IgG1 hinge domain, a transmembrane domain, a γc intracellular signaling domain, a 2B4 intracellular domain, and a CD3ζ intracellular domain. The transmembrane domain can be selected from a CD28 transmembrane domain, a CD16a transmembrane domain, a 2B4 transmembrane domain, or a NKG2D transmembrane domain.

In other embodiments, the CAR can include an extracellular antigen binding domain, a spacer, an IgG1 hinge domain, a transmembrane domain, a CD28 intracellular signaling domain, a 2B4 intracellular domain, and a CD3ζ intracellular domain. The transmembrane domain can be selected from a CD28 transmembrane domain, a CD16a transmembrane domain, a 2B4 transmembrane domain, or a NKG2D transmembrane domain.

In other embodiments, the CAR can include an extracellular antigen binding domain, a spacer, an IgG1 hinge domain, a transmembrane domain, a 2B4 intracellular signaling domain, a 41BB intracellular domain, and a CD3ζ intracellular domain. The transmembrane domain can be selected from a CD28 transmembrane domain, a CD16a transmembrane domain, a 2B4 transmembrane domain, or a NKG2D transmembrane domain.

In other embodiments, the CAR can include an extracellular antigen binding domain, a spacer, an IgG1 hinge domain, a transmembrane domain, a 2B4 intracellular signaling domain, a DAP10 intracellular domain, a DAP12 intracellular domain, and a CD3ζ intracellular domain. The transmembrane domain can be selected from a CD28 transmembrane domain, a CD16a transmembrane domain, a 2B4 transmembrane domain, or a NKG2D transmembrane domain.

In some embodiments, the extracellular antigen binding domain can include at least one polypeptide that is specific for cancer associated antigens. There are two classes of cancer associated antigens (tumor markers or antigens) that can be targeted by the CARs described herein: (1) cancer associated antigens that are expressed on the surface of cancer cells; and (2) cancer associated antigens that our themselves intracellular, however, a fragment of such antigen (peptide) is presented on the surface of the cancer cells by MHC (major histocompatability complex).

Examples of cancer associated antigens (tumor markers) include CD19, CD123, CD22, CD30, CD171, CS-1, CLL-1, CD33, EGFRvIII, GD2, GD3, BCMA, Tn Ag, PSMA, ROR1, FLT3, FAP, TAG72, CD38, CD44v6, CEA, EPCAM, B7H3, KIT, IL-13Ra2, Mesothelin, IL-11Ra, PSCA, VEGFR2, LewisY, CD24, PDGFR-beta, SSEA-4, CD20, Folate receptor alpha, ERBB2 (Her2/neu), MUC1, EGFR, NCAM, Prostase, PAP, ELF2M, Ephrin B2, IGF-I receptor, CAIX, LMP2, gp100, bcr-abl, tyrosinase, EphA2, Fucosyl GMI, sLe, GM3, TGS5, HMWMAA, o-acetyl-GD2, Folate receptor beta, TEM1/CD248, TEM7R, CLDN6, TSHR, GPRCSD, CXORF61, CD97, CD179a, ALK, Plysialic acid, PLAC1, GloboH, NY-BR-1, UPK2, HAVCR1, ADRB3, PANX3, GPR20, LY6K, OR51E2, TARP, WT1, NY-ESO-1, LAGE-1a, legumain, HPV E6, E7, MAGE-A1, MAGE A1, ETV6-AML, sperm protein 17, XAGE1, Tie 2, MAD-CT-1, MAD-CT-2, Fos-related antigen 1, p53, p53 mutant, prostein, survivin and telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, and IGLL1.

In some embodiments, the tumor antigen is a tumor antigen described in International Application PCT/US2015/020606, which is herein incorporated by reference in its entirety. In some embodiments, the tumor antigen is chosen from one or more of: CD19; CD123; CD22; CD30; CD171; CS-1 (also referred to as CD2 subset 1, CRACC, SLAMF7, CD319, and 19A24); C-type lectin-like molecule-1 (CLL-1 or CLECL1); CD33; epidermal growth factor receptor variant III (EGFRvIII); ganglioside G2 (GD2); ganglioside GD3 (aNeu5Ac(2-8)aNeu5Ac(2-3)bDGalp(1-4)bDGlcp(1-I)Cer); TNF receptor family member B cell maturation (BCMA); Tn antigen ((Tn Ag) or (GalNAca-Ser/Thr)); prostate-specific membrane antigen (PSMA); Receptor tyrosine kinase-like orphan receptor 1 (ROR1); Fms-Like Tyrosine Kinase 3 (FLT3); Tumor-associated glycoprotein 72 (TAG72); CD38; CD44v6; Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-11Ra); prostate stem cell antigen (PSCA); Protease Serine 21 (Testisin or PRSS21); vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAIX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3 (aNeu5Ac(2-3)bDGalp(1-4)bDGlcp(1-1)Cer); transglutaminase 5 (TGS5); high molecular weight-melanoma-associated antigen (HMWMAA); o-acetyl-GD2 ganglioside (OAcGD2); Folate receptor beta; tumor endothelial marker 1 (TEM1/CD248); tumor endothelial marker 7-related (TEM7R); claudin 6 (CLDN6); thyroid stimulating hormone receptor (TSHR); G protein-coupled receptor class C group 5, member D (GPRCSD); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-1a); Melanoma-associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; surviving; telomerase; prostate carcinoma tumor antigen-1 (PCTA-1 or Galectin 8), melanoma antigen recognized by T cells 1 (MelanA or MART1); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B1 (CYP1B1); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); transmembrane activator and CAML interactor (TAC1); B-cell maturation antigen (BCMA); BAFF receptor (BAFF-R); immunoglobulin lambda-like polypeptide 1 (IGLL1).

In other embodiments, the tumor antigen can include TSHR, CD171, CS-1, CLL-1, GD3, Tn Ag, FLT3, CD38, CD44v6, B7H3, KIT, IL-13Ra2, IL-11Ra, PSCA, PRSS21, VEGFR2, LewisY, CD24, PDGFR-beta, SSEA-4, MUC1, EGFR, NCAM, CAIX, LMP2, EphA2, Fucosyl GM1, sLe, GM3, TGS5, HMWMAA, o-acetyl-GD2, Folate receptor beta, TEM1/CD248, TEM7R, CLDN6, GPRCSD, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, ETV6-AML, sperm protein 17, XAGE1, Tie 2, MAD-CT-1, MAD-CT-2, Fos-related antigen 1, p53 mutant, hTERT, sarcoma translocation breakpoints, ML-IAP, ERG (TMPRSS2 ETS fusion gene), NA17, PAX3, Androgen receptor, Cyclin B1, MYCN, RhoC, CYP1B1, BORIS, SART3, PAX5, OY-TES1, LCK, AKAP-4, SSX2, CD79a, CD79b, CD72, LAIR1, FCAR, LILRA2, CD300LF, CLEC12A, BST2, EMR2, LY75, GPC3, FCRL5, and IGLL1.

In certain embodiments, the extracellular antigen binding domain comprises a polypeptide that binds to a receptor of BAFF. B-cell activating factor (BAFF) is a cytokine belonging to the tumor necrosis factor (TNF) ligand family. BAFF is abundantly produced by monocytes, macrophages, dendritic cells and stromal cells, which are main cellular components of MCL tumor microenvironment. BAFF signaling is essential for the generation of mature B cells and it helps survival of normal and malignant B cells. BAFF has at least three receptors: transmembrane activator and CAML interactor (TACI), B-cell maturation antigen (BCMA), and BAFF receptor (BAFF-R). Of these, BAFF-R is specific to BAFF while BCMA and TACI share another homologous ligand, APRIL. Signaling through BAFF-R mediates B cell survival. Virtually all mature B cell leukemias and lymphomas express BAFF receptor. Early B cells, which are counterparts of acute lymphocytic leukemia (ALL) do not express BAFF-R, but cells from patients with some cancers express high levels of BAFF-R, including ALL and mantle cell lymphoma (MCL) patients. Further, BAFF-R is expressed only on mature B cells, making it an attractive target for targeting and reducing side effects caused by off targeting. Thus, BAFF-R presents a target opportunity for treating such cancers, as well as autoimmune diseases where increased serum BAFF levels are often present.

In some embodiments, the polypeptide that binds to a receptor of BAFF can be BAFF protein or BAFF ligand. In the present disclosure, the “BAFF protein”, “BAFF ligand” or “BAFF” refers to any of the recombinant or naturally occurring forms of the B-cell activating factor or variants to homologs thereof that maintain BAFF activity (e.g., within at least about 80%, 85%, 90%, 95%, 96%, 97%, 98%, 99% or 100% activity compared to BAFF). Optionally, the variants or homologs thereof have at least about 90%, 95%, 96%, 97%, 98%, 99% or 100% amino acid sequence identity (i.e., sequence homology) wild type or naturally occurring BAFF.

In some embodiments, the BAFF ligand can include a BAFF partial sequence. A “partial sequence” or “BAFF partial sequence” refers to a portion BAFF that maintains BAFF activity similar to that of the whole sequence, and in particular, an extracellular portion of BAFF that is responsible for binding with a receptor of BAFF. In one example of a partial sequence is a sequence comprising at least 95%, 90%, 85%, 80%, 75%, 70%, 65%, 60%, 55%, 50%, 45%, 40%, 35%, 30%, 25%, 20%, 15% or 10% of the naturally occurring BAFF sequence. Also contemplated are sequences having at least 90%, 95%, 96%, 97%, 98%, 99% or 100% amino acid sequence identity across the partial sequence. In one example, the polypeptide can include a BAFF ligand that has, for example, an amino acid sequence at least about 70%, at least about 75%, at least about 80%, at least about 85%, at least about 90%, or at least about 95% identical to SEQ ID NO: 4.

In some embodiments, a BAFF-CAR can include a BAFF extracellular domain, a spacer, an IgG1 hinge domain, a transmembrane domain, a CD16a intracellular signaling domain, a 2B4 intracellular domain, and a CD3ζ intracellular domain. The transmembrane domain can be selected from a CD28 transmembrane domain, a CD16a transmembrane domain, a 2B4 transmembrane domain, or a NKG2D transmembrane domain.

In other embodiments, the BAFF-CAR can include a BAFF extracellular antigen binding domain, a spacer, an IgG1 hinge domain, a transmembrane domain, a γc intracellular signaling domain, a 2B4 intracellular domain, and a CD3ζ intracellular domain. The transmembrane domain can be selected from a CD28 transmembrane domain, a CD16a transmembrane domain, a 2B4 transmembrane domain, or a NKG2D transmembrane domain.

For example, FIG. 1 shows a schematic of two exemplary BAFF-CAR constructs with optimized intracellular domains for NK cells and an earlier BAFF-CAR construct that does not include the optimized intracellular domain. The top BAFF-CAR construct is the non-optimized construct, the middle BAFF-CAR construct is optimized using an CD16a intracellular signaling domain, and the bottom BAFF-CAR construct is optimized using a γc intracellular signaling domain.

Other embodiments described herein relate to fusion or chimeric receptor constructs that can be expressed in NK cells to improve NK cell or CAR-NK cell in vivo persistence and efficacy. The fusion receptors can convert immunosuppressive PD-1 or TIGIT signaling into the IL-21 signaling pathway. IL-21 potently reverses NK cell exhaustion and enhances in vivo persistence and antitumor efficacy. Accordingly, the fusion receptor can include a PD-1 or TIGIT extracellular domain, an IL-21R transmembrane domain, and an IL-21R intracellular signaling domain.

In some embodiments, the extracellular domain can include a PD-1 extracellular domain. The PD-1 extracellular domain can include an amino acid sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to SEQ ID NO: 46.

In other embodiments, the extracellular domain can include a TIGIT extracellular domain. The TIGIT extracellular domain can include an amino acid sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to SEQ ID NO: 44.

In some embodiments, the IL-21 transmembrane domain and intracellular domain can include an amino acid sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to SEQ ID NO: 48.

Other embodiments relate to a nucleic acid comprising a nucleotide sequence encoding a CAR and/or fusion receptor described herein. The CAR and/or fusion receptor encoding nucleotide sequence can be operably linked to a promoter and provided in an expression construct. The vector can be suitable for replication and integration into eukaryotes. Typical vectors contain transcription and translation terminators, initiation sequences, and promoters useful for regulation of the expression of the desired nucleic acid sequence.

In an embodiment, the vector is a viral vector. Viral vector technology is known in the art and is described, for example, in Sambrook et al., 2012, Molecular Cloning: A Laboratory Manual, volumes 1-4, Cold Spring Harbor Press, NY), and in other virology and molecular biology manuals. In an embodiment, viruses, which are useful as vectors are retroviruses, adenoviruses, adeno-associated viruses, herpes viruses, and lentiviruses. In an embodiment the vector is a lentivirus vector. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193)

Vectors derived from viruses, e.g., lentivirus, are suitable tools to achieve long-term gene transfer since they allow long-term, stable integration of a transgene and its propagation in daughter cells. Lentiviral vectors have the added advantage over vectors derived from retroviruses e.g., murine leukemia viruses, in that they can transduce non-proliferating cells, such as hepatocytes. They also have the added advantage of low immunogenicity.

A retroviral vector may also be, e.g., a gammaretroviral vector. A gammaretroviral vector may include, e.g., a promoter, a packaging signal (W), a primer binding site (PBS), one or more (e.g., two) long terminal repeats (LTR), and a transgene of interest, e.g., a gene encoding a CAR. A gammaretroviral vector may lack viral structural gens such as gag, pol, and env. Exemplary gammaretroviral vectors include Murine Leukemia Virus (MLV), Spleen-Focus Forming Virus (SFFV), and Myeloproliferative Sarcoma Virus (MPSV), and vectors derived therefrom.

In some embodiments, the vector can express two or more genes, where each gene is expressed separately under the control of a different promoter region, e.g., by using bi or tri-cistronic promoters. Expression of two or more genes from the same vector can be achieved by using either a multiple promoter plasmid e.g., bi or tri-cistronic promoters. Examples of multiple promoter containing lentivirus vectors are known in the literature. For example, the vector pLENTI-bi-cistronic drives the expression of two genes using the PKG promoter and the mini CMV promoter in opposite directions (Applied Biological Material Inc., Richmond, BC, Canada). Similar the tri-cistronic vector pLENTI-tri-cistronic drives expression of three genes. In this configuration one gene can be induced by the mini-CMV promoter while the second and third gene can be induced by the PGK promoter separating the two genes with a T2A peptide cleavage site.

In another embodiment, bi- or tri-cistronic vectors may also be constructed making use of internal ribosomal entry sites (IRES) such as for example the element from the encephalomyocarditis virus (EMCV) for translation of two or more open reading frames (ORFs). Such vectors are designed to drive transcription of the bi- or tri-cistronic message under control of a strong human promoter regulatory region e.g., CMV or EF1alpha. IRESs are relatively short DNA sequences that can initiate RNA translation in a 5′ cap-independent fashion. Whereas the first cistron is translated in a cap-dependent manner driven by a strong mammalian promoter, the subsequent ones utilize intercistronic regions of viral origin such as the internal ribosomal entry site of poliovirus or the cap-independent translation enhancer of encephalomyocarditis virus for enhanced translation.

Additional promoter elements, e.g., enhancers, can regulate the frequency of transcriptional initiation. Typically, these are located in the region 30-110 bp upstream of the start site, although a number of promoters have been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, the individual elements can function either cooperatively or independently to activate transcription.

Other examples of promoters include an SFFV promoter and a cytomegalovirus (CMV) promoter sequence. Other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the elongation factor-1α promoter (EF1α), the hemoglobin promoter, and the creatine kinase promoter.

Further, embodiments are not limited to the use of constitutive promoters and can include, for example, inducible promoters. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

The vector may also include, e.g., a signal sequence to facilitate secretion, a polyadenylation signal and transcription terminator (e.g., from Bovine Growth Hormone (BGH) gene), an element allowing episomal replication and replication in prokaryotes (e.g., SV40 origin and ColE1 or others known in the art) and/or elements to allow selection (e.g., puromycin resistant gene, ampicillin resistance gene and/or zeocin marker).

Sequences encoding various elements of a CAR can be disposed on the same nucleic acid molecule, e.g., the same plasmid or vector, e.g., viral vector, e.g., lentiviral vector. For example, both (i) sequence encoding extracellular antigen binding domain and (ii) sequence encoding an intracellular signaling member, can be present on the same nucleic acid, e.g., vector. Production of the corresponding proteins can be achieved, e.g., by the use of separate promoters, or by the use of a bicistronic transcription product (which can result in the production of two proteins by cleavage of a single translation product or by the translation of two separate protein products).

In order to assess the expression of a CAR polypeptide or portions thereof or fusion receptor, the vector to be introduced into an NK cell can also contain either a selectable marker gene or a reporter gene or both to facilitate identification and selection of expressing cells from the population of cells sought to be transfected or infected through viral vectors. In embodiments, the selectable marker may be carried on a separate piece of DNA and used in a co-transfection procedure. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

In some embodiments, the expression construct can further include a nucleotide sequence encoding a cytokine. The cytokine can include, for example, IL-15, IL-12, IL-2, IL-18, IL-21, or a combination thereof.

In some embodiments, the expression construct can include a nucleotide sequence that encodes the CAR and the cytokine. For example, the nucleotide sequence can be at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to SEQ ID NO: 27, the nucleotide sequence can be at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to SEQ ID NO: 28, the nucleotide sequence can be at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to SEQ ID NO: 29, the nucleotide sequence can be at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to SEQ ID NO: 30, the nucleotide sequence can be at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to SEQ ID NO: 31, the nucleotide sequence can be at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to SEQ ID NO: 40, the nucleotide sequence can be at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to SEQ ID NO: 41, or the nucleotide sequence can be at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to SEQ ID NO: 42.

In other embodiments, the expression construct can include a nucleotide sequence that encodes the fusion receptor. The expression construct encoding the fusion receptor can include a nucleotide sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to SEQ ID NO: 49, or at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to SEQ ID NO: 50.

The expression construct can be used to genetically modify, e.g., transfect or transduce, NK cells to express the CAR described herein and optional cytokine and/or fusion receptor. Methods of introducing into and expressing genes in a cell are known in the art. In the context of an expression vector, the vector can be readily introduced into a host cell, e.g., NK cell, by any method in the art. For example, the expression vector can be transferred into a host cell by physical, chemical, or biological means.

Physical methods for introducing a polynucleotide into a host NK cell include calcium phosphate precipitation, lipofection, particle bombardment, microinjection, electroporation, and the like. Methods for producing cells comprising vectors and/or exogenous nucleic acids are well-known in the art. See, for example, Sambrook et al., 2012, Molecular Cloning: A Laboratory Manual, volumes 1-4, Cold Spring Harbor Press, NY).

Biological methods for introducing a polynucleotide into a host NK cell include the use of DNA and RNA vectors as described above. Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle). Other methods of state-of-the-art targeted delivery of nucleic acids are available, such as delivery of polynucleotides with targeted nanoparticles.

NK cells genetically modified to express the CAR described herein and optional cytokine and/or fusion receptor can include Human NK cells. Human NK cells are typically characterized as lymphocytes expressing CD56 or CD16 and lacking CD3 expression, and are estimated to comprise up to about one-third of peripheral blood lymphocytes in normal subjects. Unlike T-cells, NK cells recognize targets in a major histocompatibility complex (MHC)-unrestricted manner. There are various known methods for isolating NK cells from peripheral blood. Generally, to isolate natural killer cells from peripheral blood, PBMCs are separated into lymphocytes and monocytes, and the lymphocytes are further divided into T cells, B cells, and natural killer cells for isolation.

The peripheral blood mononuclear cells can be obtained from human blood collected using known methods such as the Ficoll-Hypaque density gradient method. PMBCs may be obtained from a healthy person, a patient at risk of cancer, or a cancer patient. The PBMCs used herein can be, but do not necessarily need to be, autologous; allogeneic PBMCs may also be used to induce and proliferate the NK cells for anti-cancer immunotherapy according to the present disclosure.

In some embodiments, NK cells may be derived from a subject and grown in vitro to provide a population of NK cells for use in the present disclosure. In deriving NK cells from a subject, the cells may come from stem cells or they may be collected from a living donor. In a preferred aspect, the NK cells employed herein are collected from a living donor. In certain aspects, the living donor many be a human living donor. In an alternative embodiment, a NK cell known in the art that has previously been isolated and cultured may be used in the present invention. Thus, an established NK cell line may be used. Many such NK cells lines are commercially available and known to those in the art.

Once isolated, NK cells can be expanded if larger numbers are desired. As used herein, “expanded” refers to the increase in number of NK cells by any method. Though several expansion platforms have been developed for NK cells, few have the potential to efficiently produce a large magnitude of highly active NK cells.

In some embodiments, the administration of the NK cell may be non-immunogenic, for example, by providing a conditioning regimen (e.g., cyclophosphamide and fludarabine) to the patient at the time of administration. The term “non-immunogenic” is thus used broadly herein to mean that when the cell is injected into or otherwise administered to a subject, it avoids detection by the body's immunological system and is not rejected or recognized as foreign. More particularly, the cell does not raise (or is not capable of raising) an immune response sufficient to lead to rejection of the cell and/or to affect the function of the cells. Thus, the cells retain cytotoxic activity in the subject, more particularly, significant or substantial or measurable cytotoxic activity against a target cell. As with any biological system, the absence of an immune response may not be absolute (or 100%), A small (or mild or minor) immune response to the NK cell (e.g., a de minimis immune response) may be tolerated, as long as the function or utility of the cells is not substantially affected (i.e., as long as the cells can still perform their function). That is, the NK cells employed herein may be “universal” in nature such that one set of donor cells can be used for virtually any patient without generating a negative immune response.

The NK cells may be autologous or allogeneic NK cells. If the NK cells are derived from an identical twin, they may be termed “syngeneic”. In particularly preferred embodiments, the NK cells employed according to the disclosed methods, including the methods of treating cancer, are autologous or allogeneic NK cells.

In some embodiments, the isolated NK cells can be expanded using feeder cells. As used herein, the term “feeder cells” refers to cells that, due to their metabolic activity, produce various metabolites to thereby assist in the proliferation of target cells, even though these cells cannot themselves proliferate.

Feeder cells as used according to the present disclosure may be any population of leukemia cells engineered to express a membrane-bound interleukin (mbIL). As used herein, the term “interleukin (IL) protein” refers to a collection of biologically active cytokines produced by immune cells such as lymphocytes, monocytes or macrophages. According to the present disclosure, the term “cytokine” refers to an immune activating cytokine (secreted protein and/or signaling molecule) that can be used to induce NK cells isolated from PBMCs. Examples of IL proteins which may be used in the present disclosure include IL-2, IL-7, IL-12, IL-15, IL-18, IL-21, Flt3-L, SCF, IL-7 and the like.

In certain aspects of the disclosure, the feeder cells are HL-60 cells or OCI-AML3 cells. In a preferred aspect, the feeder cells are OCI-AML3 cells. Preferably, the mbIL comprises one or more of IL-15 or IL-21. In a preferred aspect, the mbIL consists of, or consists essentially of, mbIL-15. In another preferred aspect, the mbIL consists of, or consists essentially of, mbIL-21.

The cells that are used as feeder cells may be non-inactivated or inactivated cells whose proliferation was inhibited prior to use. More specifically, the feeder cells may be inactivated to ensure their safety and to eliminate their potential to proliferate when employed as part of the feeding platform described herein. A common method for inactivating feeder cells comprises the step of irradiating the killer cells with gamma-rays. If non-inactivated feeder cells are used, they can be killed by natural killer cells during culture because they are tumor cells. In a preferred aspect of the present disclosure, the feeder cells are inactivated using gamma radiation prior to adding them to the cell culture comprising NK cells. In some embodiments, the feeder cells can be inactivated using 10 Gy or greater or of radiation, such as 10 Gy, 20 Gy, 30 Gy, 40 Gy, 50 Gy, 60 Gy, 70 Gy, 80 Gy, 90 Gy, 100 Gy, 110 Gy, 120, Gy, 130 Gy, 140 Gy, 150 Gy, 175 Gy, 200 Gy, 225 Gy, 250 Gy, 300 Gy, 350 Gy, 400 Gy, 450, Gy or 500 Gy. In preferred methods, the feeder cells are inactivated using 90 Gy of gamma radiation.

In some embodiments, ex vivo expansion of the NK cells employs a feeder cell line constructed from an AML cells line transduced with a membrane-bound IL protein. In some aspects of the present disclosure, OCI-AML3 cells, HL-60 cells, or combinations thereof, are employed as the feeder cell line. Preferably, OCI-AML3 cells are employed as the feeder cell line.

The presence of membrane-bound IL proteins, such as membrane-bound IL-15 (mbIL-15) or membrane-bound IL-21 (mbIL-21) is believed to prevent NK cells from undergoing senescence, markedly improving their ability to expand ex vivo. There are a myriad of studies proposing how and why cytokines exert their effect on NK cell function and prevention of senescence, but a single, clear explanation has not yet emerged. The feeder cells of the present disclosure preferably comprise mbIL-15, mb-IL-21 or combinations thereof as the membrane-bound IL protein.

In some embodiments, the feeder cell line has been engineered to express mbIL-15 and/or mbIL-21. The feeder cells according can be further modified to express one or more associated accessory signaling polypeptides, cytokines or fragments thereof. Such expression may correlate with increased expression of the mbIL proteins in certain aspects.

In addition to the NKF cells, NK cells can be expanded in vivo or ex vivo in the presence of cytokine support. Cytokine support can be used to enable the cells to survive and proliferate after infusion into the patient. Exemplary cytokines for use according to the disclosed methods include IL-2, IL-15, ALT-803, hetIL-15, IL-12, IL-18, IL-21 or fragments or derivatives thereof. The present methods may comprise the use of more than one cytokine support. In preferred aspects of the present methods, NK cells may be expanded in the presence of IL-2, IL-15 or ALT-803 or other IL-15 derivatives. Alternatively, cytokine support can be provided by engineering the NK cells to express additional cytokines. For example, this can be accomplished by transducing genes for one or more of mbIL-15, soluble IL-15, soluble IL-21, mbIL-21, mbIL-2, or soluble IL-2 into the NK cells prior to or after NK cell expansion. In some aspects of the disclosure, cytokine support is provided by pharmacologic inhibitors. In some aspects of the disclosure, cytokine support is provided by genetic modification. In other aspects, cytokine support can be provided both pharmacologically and genetically.

In certain aspects of the disclosed methods, the expanding of NK cells in the presence of NKF cells can last up from one to eight weeks. That is, the step of expanding NK cells can take, e.g., one week, two weeks, three weeks, four weeks, five weeks, six weeks, seven weeks, or eight weeks.

When NK cells are expanded for more than one week, the NKF cells may need refreshed or replenished throughout the step of expanding the NK cells. Refreshing of NKF cells can be done on an as-needed basis, preferably weekly. The amount of NKF cells for refreshment can employ the same ratio of NKF:NK cells as the starting ratio or the NKF cells can be replenished in a different ratio as needed. In some aspects of the present method, the ratio of NKF cells to NK cells of refreshment is preferably greater than or equal to about 1:1, such as about 1:1, about 1.5:1, about 2:1, about 2.5:1, about 3:1, about 3.5:1, about 4:1, about 4.5:1, about 5:1, about 5.5:1, about 6:1, about 6.5:1, about 7:1, about 7.5:1, about 8:1, about 8.5:1, about 9:1, about 9.5:1, or about 10:1, based on the number of NK cells counted on the day of the NKF cell addition. In some aspects, a 5:1 ratio of NKF:NK cells is particularly preferred. In other aspects of the present method, the NKF:NK cell ratio is about 10:1, or greater, such as 10:1, 15:1, 20:1, 25:1 or 30:1, based on the NK cell count on the day of the NKF cell addition. In a particularly preferred embodiment, the NKF:NK cell ratio is 10:1.

In some embodiments, at least about 40%, at least about 50%, at least about 60%, at least about 70%, at least about 80%, or at least about 90% of the enriched population of immune cells are NK cells genetically modified to express the CAR and one or more proteins capable of providing cytokine support. The one or more proteins capable of providing cytokine support are selected from mbIL-15, soluble IL-15, soluble IL-21, mbIL-21, mb-IL-2, or soluble IL-2. In other embodiments, at least about 40%, at least about 50%, at least about 60%, at least about 70%, at least about 80%, or at least about 90% of the immune cells are NK cells are genetically modified to express the CAR described herein and one or more proteins capable of inhibiting TGFβ signaling.

In some embodiments, a plurality of the engineered NK cells can be provided in an immunotherapy composition. The immunotherapy composition can be used in a method of treating cancer in a subject in need thereof. The method includes administering to a subject a therapeutically effective amount of immunotherapy composition comprising the engineered NK cells thereby treating cancer in the subject.

In some embodiments, the immunotherapy composition administered to the subject can include at least about 1 million engineered CAR-NK cells, at least about 2 million engineered CAR-NK cells, at least about 3 million engineered CAR-NK cells, at least about 4 million engineered CAR-NK cells, at least about 5 million engineered CAR-NK cells, or at least about 10 million engineered CAR-NK cells.

In another aspect, a method of treating cancer in a subject in need thereof is provided including administering to a subject a therapeutically effective amount of the immunotherapy composition provided herein, thereby treating cancer in the subject. Optionally, the cancer is lymphoma, leukemia or myeloma. Optionally, the cancer is lymphoma. Optionally, the lymphoma is mantle cell lymphoma, follicular lymphoma, diffuse large B-cell lymphoma, marginal zone lymphoma or Burkitt's lymphoma. Optionally, the lymphoma is mantle cell lymphoma. Optionally, the lymphoma is follicular lymphoma. Optionally, the lymphoma is diffuse large B-cell lymphoma. Optionally, the lymphoma is marginal zone lymphoma. Optionally, the lymphoma is Burkitt's lymphoma.

Optionally, the cancer is leukemia. Optionally, the leukemia is lymphoblastic leukemia, chronic lymphocytic leukemia or hairy cell leukemia. Optionally, the leukemia is lymphoblastic leukemia. Optionally, the leukemia is chronic lymphocytic leukemia. Optionally, the leukemia is hairy cell leukemia.

Optionally, the cancer is myeloma. Optionally, the myeloma is multiple myeloma.

Optionally, the method further includes administering to the subject an additional therapeutic in combination with the immunotherapy composition. The additional therapeutic can include other types of therapy for cancer, such as chemotherapy, surgery, radiation, gene therapy, and so forth. Such therapies can be administered simultaneously or sequentially (in any order) with the immunotherapy composition described herein. When co-administered with an additional therapeutic, suitable therapeutically effective dosages for each agent may be lowered due to the additive action or synergy.

Non-limiting examples of other anti-cancer therapeutic agents useful for combination with the modified immune cells described herein include, but are not limited to, immune checkpoint inhibitors (e.g., PDL1, PD1, and CTLA4 inhibitors), anti-angiogenic agents (e.g., TNP-470, platelet factor 4, thrombospondin-1, tissue inhibitors of metalloproteases, prolactin, angiostatin, endostatin, bFGF soluble receptor, transforming growth factor beta, interferon alpha, soluble KDR and FLT-1 receptors, and placental proliferin-related protein); a VEGF antagonist (e.g., anti-VEGF antibodies, VEGF variants, soluble VEGF receptor fragments); chemotherapeutic compounds. Exemplary chemotherapeutic compounds include pyrimidine analogs (e.g., 5-fluorouracil, floxuridine, capecitabine, gemcitabine and cytarabine); purine analogs (e.g., fludarabine); folate antagonists (e.g., mercaptopurine and thioguanine); antiproliferative or antimitotic agents, for example, vinca alkaloids; microtubule disruptors such as taxane (e.g., paclitaxel, docetaxel), vincristin, vinblastin, nocodazole, epothilones and navelbine, and epidipodophyllotoxins; DNA damaging agents (e.g., actinomycin, amsacrine, anthracyclines, bleomycin, busulfan, camptothecin, carboplatin, chlorambucil, cisplatin, cyclophosphamide, cytoxan, dactinomycin, daunorubicin, doxorubicin, epirubicin, hexamethyhnelamineoxaliplatin, iphosphamide, melphalan, merchlorehtamine, mitomycin, mitoxantrone, nitrosourea, plicamycin, procarbazine, taxol, taxotere, teniposide, triethylenethiophosphoramide and etoposide).

In some embodiments, radiation, or radiation and chemotherapy are used in combination with the cell populations comprising modified immune cells described herein. Additional useful agents and therapies can be found in Physician's Desk Reference, 59.sup.th edition, (2005), Thomson P D R, Montvale N.J.; Gennaro et al., Eds. Remington's The Science and Practice of Pharmacy 20.sup.th edition, (2000), Lippincott Williams and Wilkins, Baltimore Md.; Braunwald et al., Eds. Harrison's Principles of Internal Medicine, 15.sup.th edition, (2001), McGraw Hill, N.Y.; Berkow et al., Eds. The Merck Manual of Diagnosis and Therapy, (1992), Merck Research Laboratories, Rahway N.J.

Also provided are methods of treating an autoimmune disease in a subject in need thereof. The method includes administering to the subject a therapeutically effective amount of the immunotherapy composition as provided herein, thereby treating an autoimmune disease in the subject. Optionally, the autoimmune disease is rheumatoid arthritis, systemic Lupus erythematosus, multiple sclerosis, glomerulonephritis, Sjögren's Syndrome or autoimmune hemolytic anemia. Optionally, the autoimmune disease is rheumatoid arthritis. Optionally, the autoimmune disease is systemic Lupus erythematosus. Optionally, the autoimmune disease is multiple sclerosis. Optionally, the autoimmune disease is glomerulonephritis. Optionally, the autoimmune disease is Sjögren's Syndrome. Optionally, the autoimmune disease is autoimmune hemolytic anemia. Optionally, the method further includes administering to the subject a second therapeutic agent.

The following examples are illustrative and are not intended to limit the scope of the invention as claimed.

### Example 1

Chimeric Antigen Receptor Constructs with Optimized Intracellular Domains for Natural Killer Cells

In this example, we describe two novel CAR constructs that we designed specifically for implementation in NK cells. Their novelty lies in the first intracellular signaling domain that follows the CD28 transmembrane domain. One construct utilizes the intracellular signaling domain of CD16A, while the other utilizes the intracellular signaling domain of the common gamma chain, or γc (FIG. 1). To our knowledge, no one has yet reported the design or testing of CAR constructs that utilize either of these intracellular domains. In this example, the intracellular signaling domain of CD16 is fused to the other domains of the CAR construct at both its N-terminus and C-terminus and is designed to enhance intracellular signaling elicited by the CAR construct when stimulated by BAFF binding to BAFF receptor on the target cancer cell.

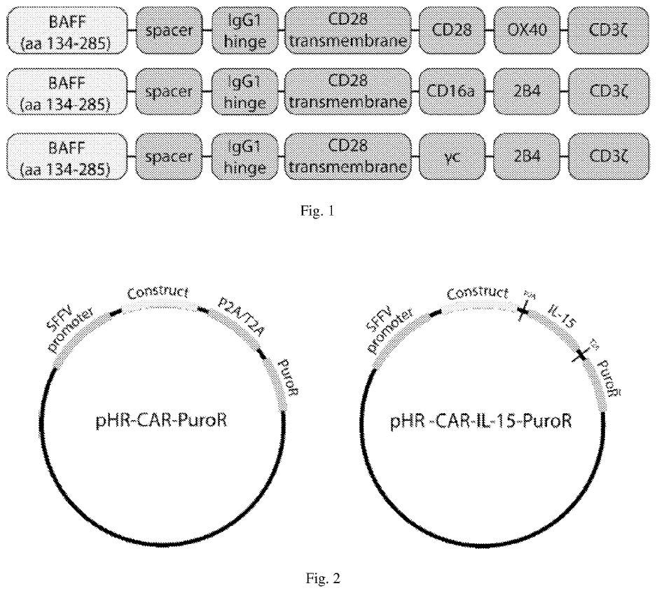

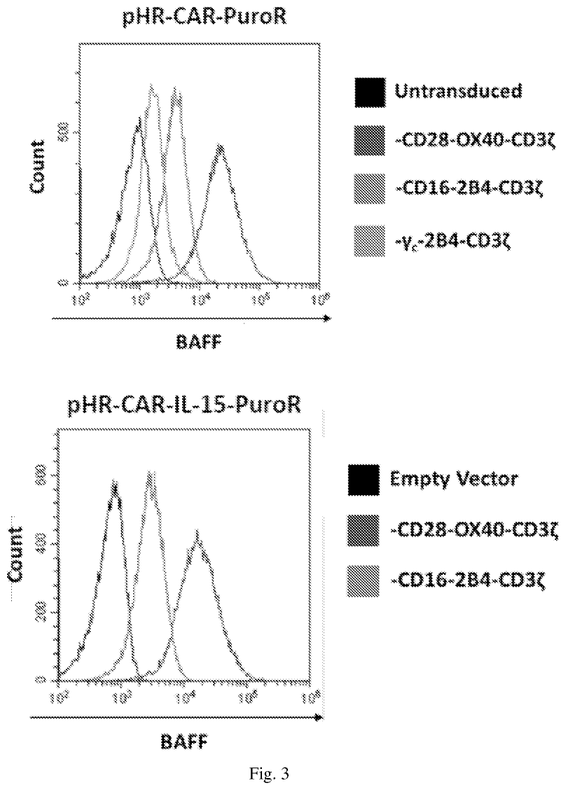

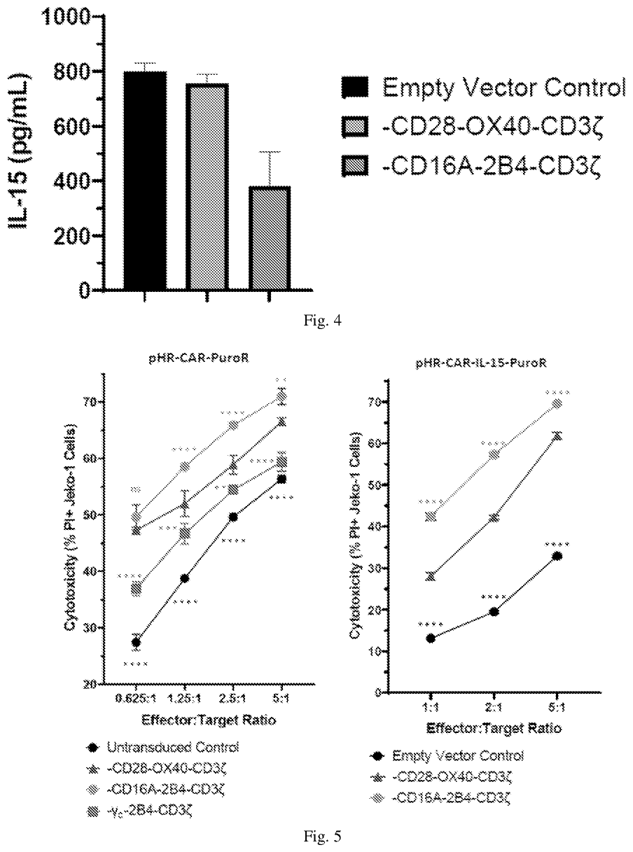

Using the pHR lentiviral expression vector, we produced lentivirus containing the respective BAFF-CAR nucleotide sequence to transduce NK92 cells, a human NK cell line (FIG. 2). Successfully transduced cells were positively selected using a puromycin resistance gene downstream of the BAFF-CAR construct. For select constructs, we used the pHR-CAR-IL-15-PuroR vector, which codes for both the CAR construct and IL-15 secretion (FIG. 2). CAR-NK cells were stained for BAFF surface expression and analyzed using flow cytometry to confirm transduction and surface expression of the BAFF-CAR construct (FIG. 3). NK cells transduced with the pHR-CAR-IL-15-PuroR vector were cultured for 72 h, and IL-15 secretion was confirmed via ELISA (FIG. 4). Using in vitro cytotoxicity assays against the Jeko-1 mantle cell lymphoma cell line, we tested the efficacy of our novel CAR constructs relative to the original BAFF-CAR construct (−CD28-OX40-CD3ζ) that we previously reported. BAFF CAR-NK cells with the −CD16A-2B4-CD3ζ construct displayed significantly improved cytotoxicity compared to cells with the original construct or untransduced or empty vector controls (FIG. 5). This was true whether the cells were engineered to secreted IL-15 or not. BAFF CAR-NK cells with the −γ_{c}-2B4-CD3ζ construct had significantly higher cytotoxicity than untransduced control, but its cytotoxicity was still lower than that of NK cells with the −CD28-OX40-CD3ζ or −CD16A-2B4-CD3ζ construct.

Thus, we have functionally validated the efficacy of novel CAR constructs specifically designed for NK-based cancer therapy. Specifically, the −CD16A-2B4-CD3ζ construct exhibits superior efficacy than our previously published construct. We anticipate this to translate into improved CAR-NK efficacy, regardless of whether the cells are designed to target BAFF receptors (BAFF-CAR) or other antigens.

### Example 2

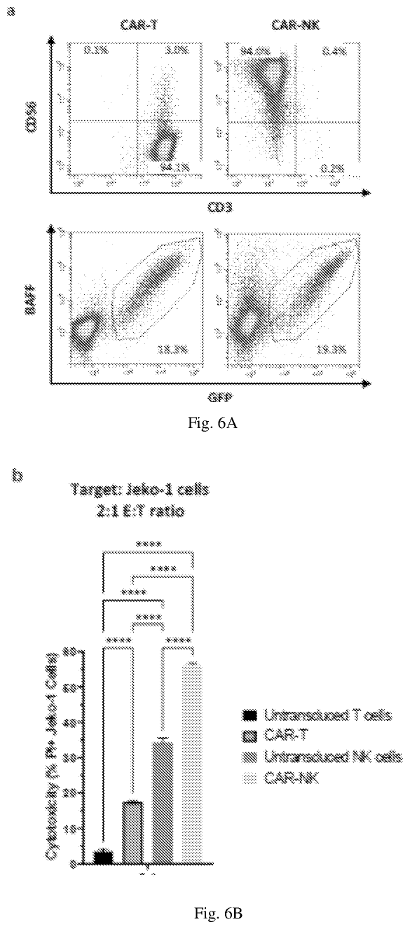

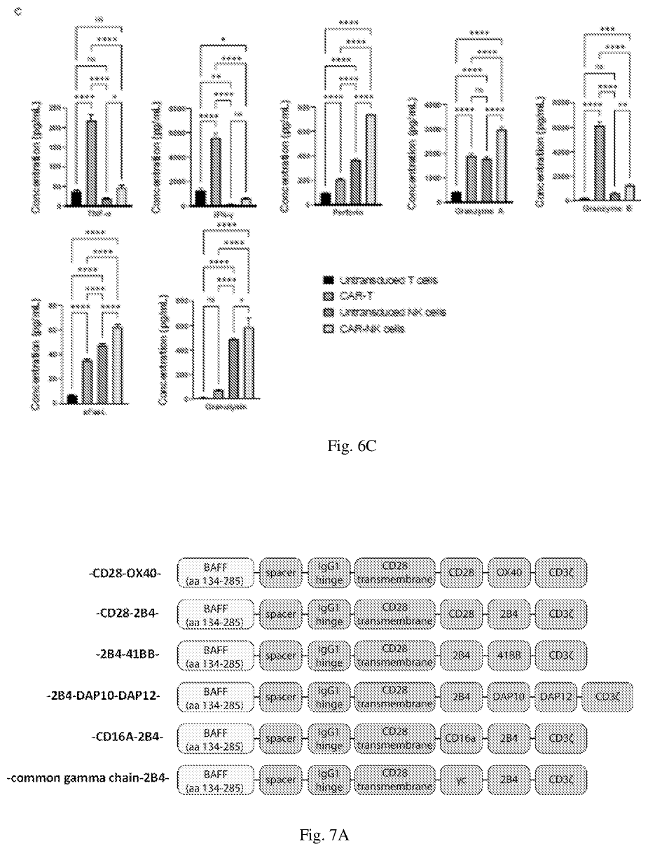

Lentiviral transduction of primary (1°) human NK cells from healthy donors is much more challenging than transduction of 1° T cells or the NK92 cell line, but we have optimized our protocol to yield ~20% transduction efficiency (FIG. 6A). Using T cells that were transduced with the same CAR+ percentage, we conducted a 16-hour cytotoxicity experiment using the Jeko-1 mantle cell lymphoma (MCL) cell line. We observed significantly higher cytotoxicity from BAFF CAR-NK cells compared to untransduced NK cells, as well as against BAFF CAR-T cells and untransduced T cells (FIG. 6B). Co-culture supernatant from the assay was collected to assess cytokine release, as one of the translational/clinical advantages of CAR-NK over CAR-T therapy is the reduced release of pro-inflammatory cytokines that trigger cytokine release syndrome (CRS) and life-threatening adverse effects in patients. Under the same experimental conditions, compared to BAFF CAR-T cells, the BAFF CAR-NK cells secreted significantly lower levels of TNF-α (47 vs 218 μg/mL) and IFN-γ (654 vs 5,575 pg/mL), the principal cytokines released by cytotoxic cells that activate monocytes and trigger CRS (FIG. 6C). Meanwhile, BAFF CAR-NK cells still secrete higher levels of perforin and granzyme A, likely explaining the higher in vitro cytotoxicity that we observed.

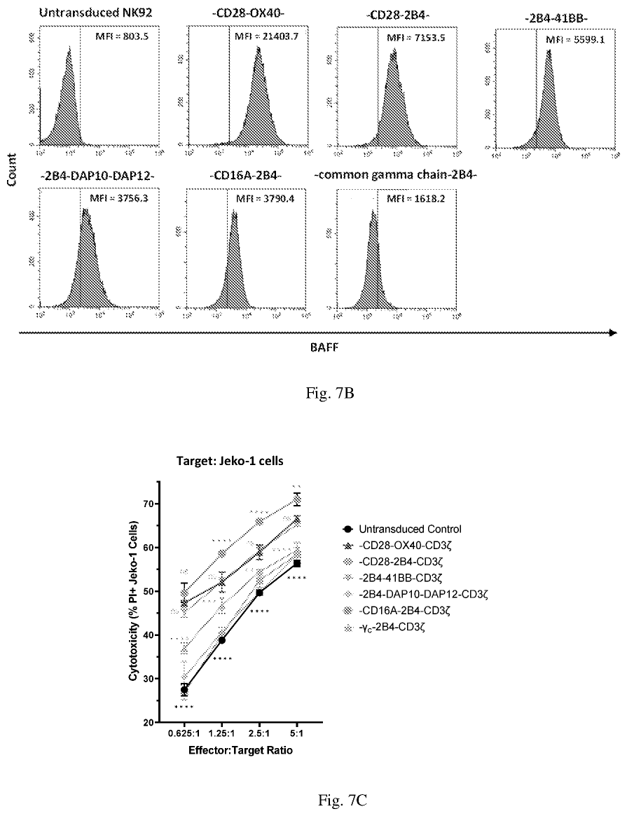

Alongside the optimizations required of working with 1° NK cells, we tested novel, NK-specific CAR constructs that would further enhance efficacy. The intracellular domains of the original CAR construct was based on CAR-T designs, and based on the literature, we believed that our construct would benefit greatly from the introduction of signaling domains that have greater prominence in NK cells (FIG. 7A). These include the intracellular domains of 2B4, DAP10, and DAP12, as well as of CD16A and the common gamma chain (γc), which have not been previously investigated for CAR-NK intracellular signaling. We outsourced the nucleotide synthesis of these constructs and subcloned their sequences into our lentiviral construct, then transduced NK92 cells and selected CAR+ cells via puromycin selection. Surface expression of the BAFF-CAR construct was confirmed using flow cytometry (FIG. 7b). These BAFF CAR-NK cells were mixed with Jeko-1 cells at different effector:target ratios in a 16 h cytotoxicity experiment, and of the 5 new constructs tested, the CD16A-2B4 construct demonstrated significantly superior cytotoxicity (65.9% at 2.5:1 ratio) to the original CD28-OX40 construct (58.9%) and the untransduced control (49.7%) (FIG. 7C). This represents a 12% improvement over the original construct, which itself displayed 18% greater cytotoxicity than unmodified NK92 cells. Overall, our CD16A-2B4 displays 33% higher target cell killing compared to the negative control. The γc-2B4 construct demonstrated higher cytotoxicity than untransduced cells, but lower cytotoxicity than the original construct, while the 2B4-41BB construct proved similar in efficacy to the original. The remaining 2 constructs did not appear to be any different than the untransduced control.

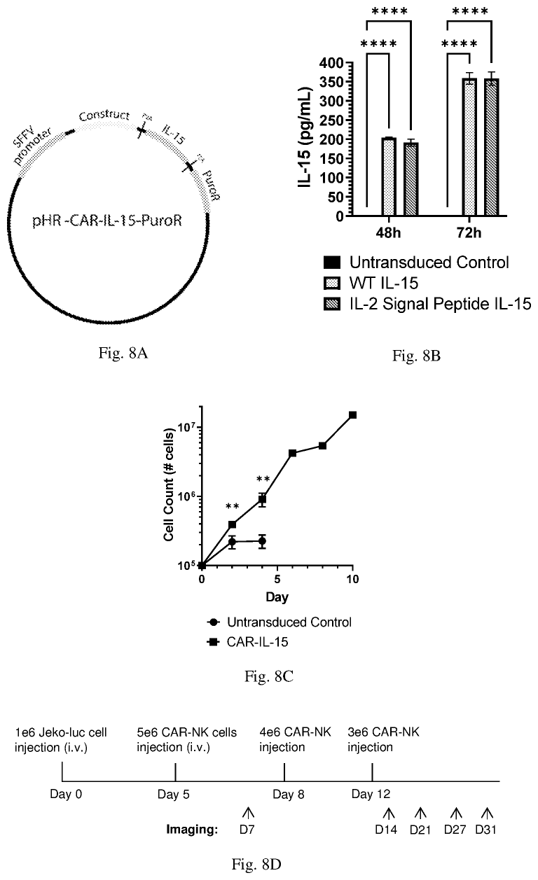

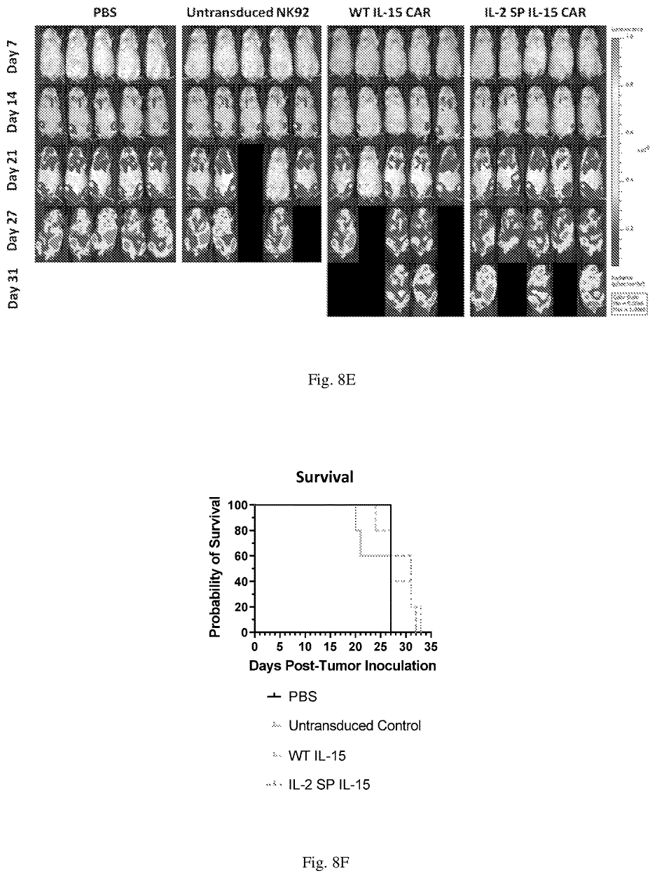

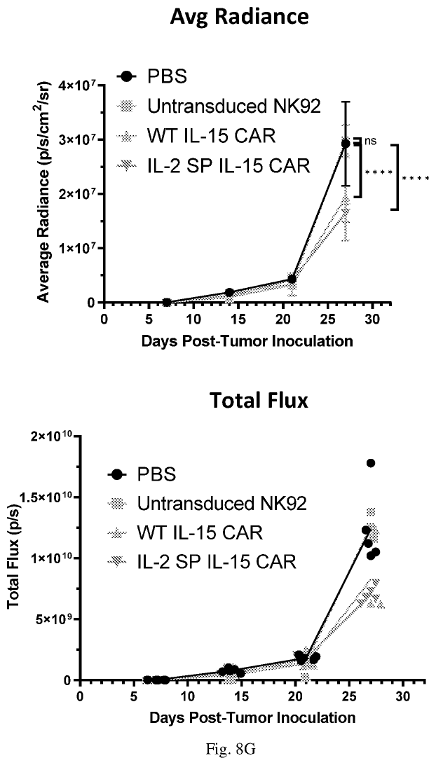

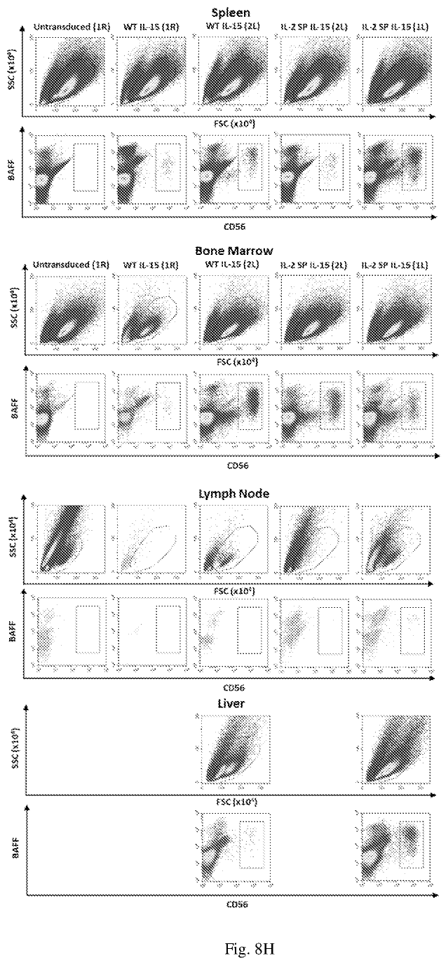

For in vivo efficacy experiments, we tested our original CD28-OX40 BAFF-CAR-NK92 cells in vivo using luminescent Jeko-1 cells (Jeko-luc). The literature has demonstrated the improved in vivo persistence of CAR-NK cells that simultaneously secrete IL-15. We wondered whether we could improve upon the original idea by substituting the wild-type IL-15 signal peptide (WT IL-15) with the IL-2 signal peptide, as the literature has reported that IL-2 is secreted with higher efficiency than IL-15 by cells that naturally produce these cytokines. Therefore, we subcloned both of these human IL-15 sequence into our lentiviral vector downstream of the BAFF-CAR transgene (FIG. 8A). We produced BAFF-CAR NK92 cells and confirmed IL-15 secretion in vitro, but we did not see any difference in IL-15 secretion between WT and IL-2 signal peptides (FIG. 8B). We demonstrated that WT IL-15 CAR-NK92 cells are capable of sustaining their own survival and expansion in cell culture, as opposed to untransduced NK92 cells, which die off without the external provision of IL-2 or IL-15 (FIG. 8C). Finally, we used these cells in an in vivo experiment. Following intravenous (i.v.) injection of 1e6 Jeko-luc cells, 3 rounds of CAR-NK injection were conducted i.v., and imaging was performed on the indicated days (FIG. 8D). Although the CAR-NK92 cells displayed prolonged survival that was clear through bioluminescence imaging, this improvement in survival was not statistically significant (FIGS. 8E-G). This was not entirely unexpected, as in the literature, the majority of successful NK experiments in vivo have used 1° NK cells vs NK92 cells in vivo. We were able to detect CAR-NK92 cell persistence, but not untransduced NK92 persistence, in various organs of the treated mice, including spleen, bone marrow, liver, and some cervical lymph nodes (FIG. 8H).

### Example 3

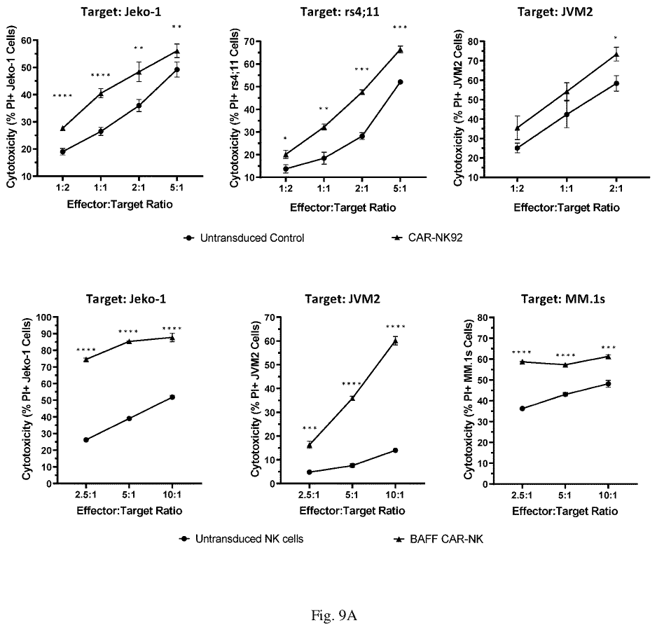

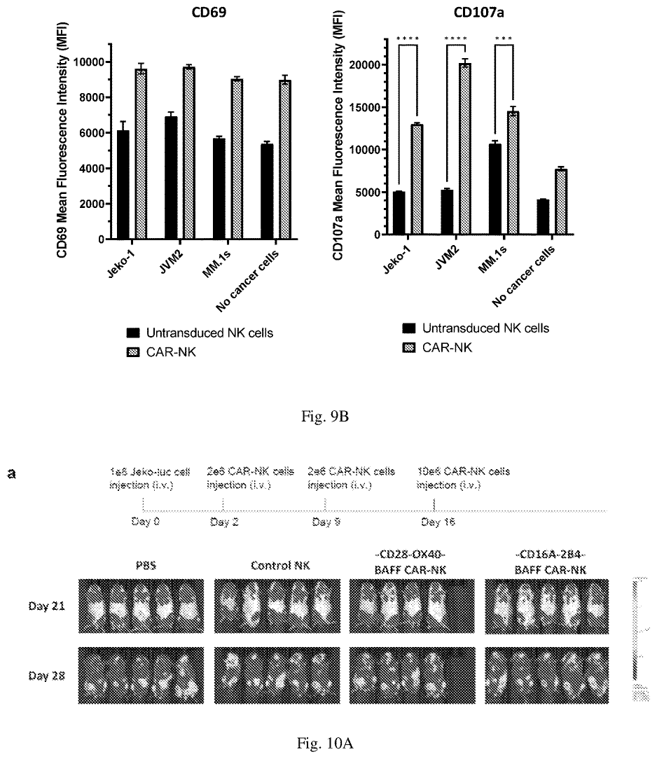

We demonstrated significant in vitro cytotoxicity of 1° BAFF CAR-NK cells against JVM2, a mantle cell lymphoma (MCL) cell line, and MM.1s, a multiple myeloma (MM) cell line (FIG. 9A); previously, our in vitro characterization using 1° NK cells was largely limited to Jeko-1 MCL cells. Compared to untransduced NK cells, 10 BAFF CAR-NK cells exhibit up to 40%, 50%, and 25% greater cytotoxicity against Jeko-1, JVM2, and MM.1s cells, respectively (FIG. 9A). Notably, this difference in cytotoxicity against Jeko-1 cells is higher than any previous experiment, likely due to improved transduction efficiency. Additionally, we stained these NK cells for the degranulation marker CD107a, something we could not do with NK92 cells since the cell line does not significantly express this marker at the cell surface. We observed significantly higher CD107a expression by 1° BAFF CAR-NK cells compared to untransduced NK cells when co-cultured with these different cancer cells (FIG. 9B). While untransduced NK cells do not noticeably increase CD107a expression when co-cultured with Jeko-1 or JVM2 compared to no cancer cells, BAFF CAR-NK cells have much higher CD107a expression. This reflects the increased and specific activation of the CAR-NK cells via the BAFF-CAR construct binding to BAFF receptors present on the target cells. This difference in CD107a was most noticeable with the JVM2 cells, which correlates with the significant cytotoxicity seen in FIG. 9A. Interestingly, both untransduced NK cells and BAFF CAR-NK cells display increased degranulation when co-cultured with MM.1s cells (FIG. 9B); this may suggest an elevated basal NK cell sensitivity to this myeloma cell line. As such, lower effector:target (E:T) ratios may better capture the cytotoxicity advantage of the BAFF-CAR construct, as higher E:T ratios would result in significant cytotoxicity against the MM. Is cells by both BAFF CAR-NK cells and untransduced NK cells (FIG. 9A).

Using the improved transduction protocol, we again conducted an in vivo cytotoxicity experiment using the intravenous (i.v.) Jeko-1 xenograft mouse model. We believed that 1° BAFF CAR-NK cells derived from human peripheral blood would be superior to the NK92 cells previously used. We also wanted to observe whether our novel CD16A-2B4 BAFF-CAR construct, which demonstrated superior cytotoxicity to the original CD28-OX40 construct as described in the previous annual report, would translate to improved in vivo efficacy.

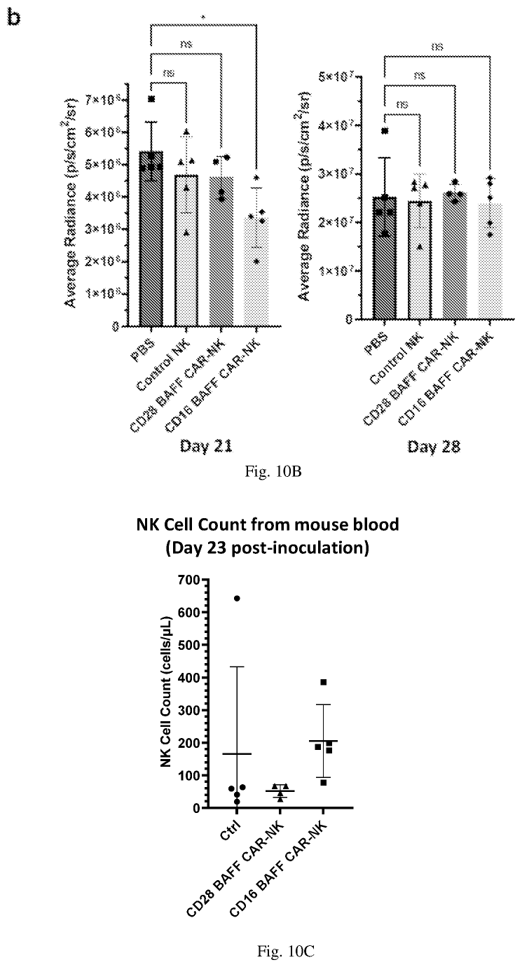

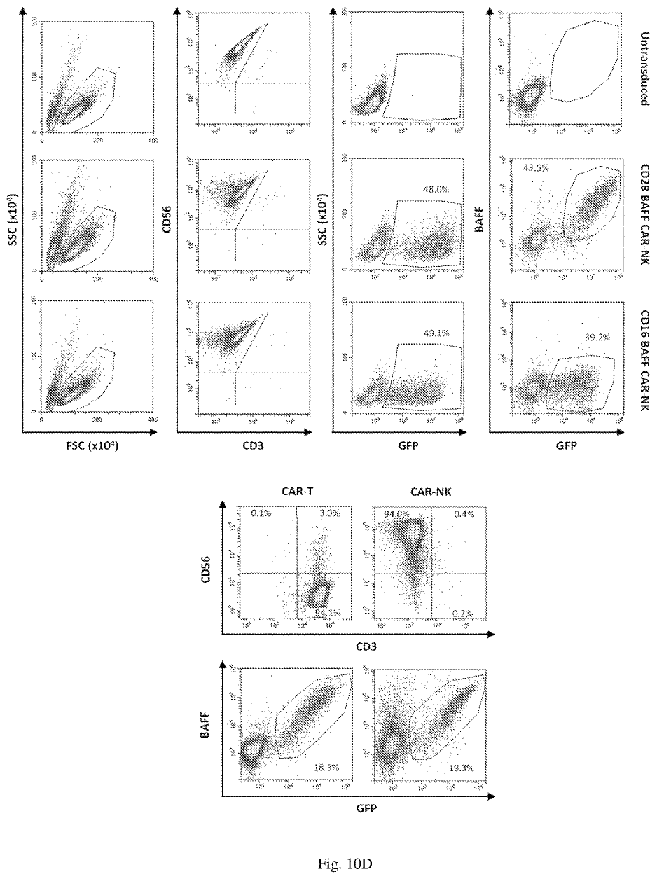

1e6 luciferase-expressing Jeko-1 cells (Jeko-1-luc) were injected via tail vein, followed by three total treatments administered i.v. 7 days apart from each other (FIG. 10A). Tumor engraftment and growth were tracked via bioluminescence imaging over 4 weeks, and the imaging from Days 21 and 28 post-tumor inoculation are shown here (FIG. 10A). Interestingly, the mice treated with CD16 BAFF CAR-NK cells displayed a lower level of tumor burden at Day 21 than was statistically significant but not the mice treated with the original CD28 BAFF-CAR construct (FIGS. 10A, B). On Day 23, more human NK cells were detectable in the peripheral blood of mice treated with CD16 BAFF CAR-NK cells compared to CD28 BAFF CAR-NK or Control NK cells (FIG. 10C). However, by Day 28, any difference in tumor burden among any of the treatment groups was no longer discernible (FIG. 10B). These results suggest that the CD16A-2B4 BAFF-CAR construct, which we built specifically for use in NK cells, may indeed be superior to the original construct, which was originally used in T cells.

### Example 4

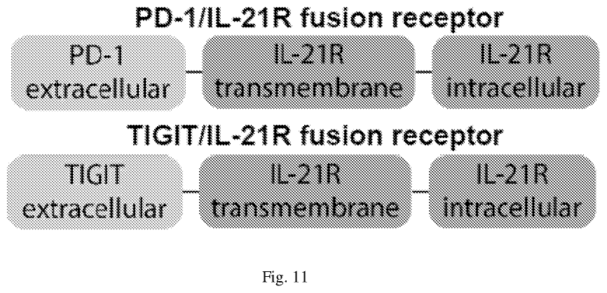

To improve CAR-NK in vivo persistence and efficacy, we designed novel fusion receptor proteins that convert immunosuppressive PD-1 or TIGIT signaling into the IL-21 signaling pathway (FIG. 11). IL-21 potently reverses NK cell exhaustion and enhances in vivo persistence and antitumor efficacy. Similar fusion concepts (IL-4/IL-21, PD-1/CD28) have been successfully tested in CAR-T cells, but almost none have been tested in CAR-NK cells. Given the prominence of TIGIT in marking dysfunctional NK cells, our PD-1/IL-21R and TIGIT/IL-21R fusion receptors are uniquely designed to prevent CAR-NK exhaustion, improve their efficacy and be incorporated with our BAFF-CAR construct to enhance its overall performance.

The fusion receptor can be incorporated into the expression plasmid containing our BAFF-CAR construct. A tandem P2A/T2A self-cleaving peptide sequence can be used to separate their translation. Then, in vitro and in vivo experiments can be used to demonstrate that both the BAFF-CAR construct and the fusion receptors remain functional. It is expected that their combination to significantly prolong mice survival compared to mice treated with just BAFF CAR-NK cells or just NK cells expressing the fusion receptors alone.

### Example 5

CD8α signal peptide
(SEQ ID NO: 1)
atggccttaccagtgaccgccttgctcctgccgctggccttgctgctccacgccgccaggccg
(SEQ ID NO: 2)
MALPVTALLLPLALLLHAARP
Extracellular BAFF (amino acids 134-285)
(SEQ ID NO: 3)
gccgtgcagggccccgaggagaccgtgacccaggactgcctgcagctgatcgccgacagcgagacccccaccatccagaaggg
cagctacaccttcgtgccctggctgctgagcttcaagaggggcagcgccctggaggagaaggagaacaagatcctggtgaaggag
accggctacttcttcatctacggccaggtgctgtacaccgacaagacctacgccatgggccacctgatccagaggaagaaggtgcac
gtgttcggcgacgagctgagcctggtgaccctgttcaggtgcatccagaacatgcccgagaccctgcccaacaacagctgctacagc
gccggcatcgccaagctggaggagggcgacgagctgcagctggccatccccagggagaacgcccagatcagcctggacggcga
cgtgaccttcttcggcgccctgaagctgctg
(SEQ ID NO: 4)
AVQGPEETVTQDCLQLIADSETPTIQKGSYTFVPWLLSFKRGSALEEKENKILVKETG
YFFIYGQVLYTDKTYAMGHLIQRKKVHVFGDELSLVTLFRCIQNMPETLPNNSCYSA
GIAKLEEGDELQLAIPRENAQISLDGDVTFFGALKLL
Spacer
(SEQ ID NO: 5)
tctggaggcggctcggatccc
(SEQ ID NO: 6)
SGGGSDP
IgG1 Hinge
(SEQ ID NO: 7)
gccgagcccaaatctcctgacaaaactcacacatgcccaccgtgcccaaaagatcccaaa
(SEQ ID NO: 8)
AEPKSPDKTHTCPPCPKDPK
CD28 Transmembrane
(SEQ ID NO: 9)
ttttgggtgctggtggtggttggtggagtcctggcttgctatagcttgctagtaacagtggcctttattattttctgggtg
(SEQ ID NO: 10)
FWVLVVVGGVLACYSLLVTVAFIIFWV
Intracellular CD16A
(SEQ ID NO: 11)
aagacaaacattcgaagctcaacaagagactggaaggaccataaatttaaatggagaaaggaccctcaagacaaa
(SEQ ID NO: 12)
KTNIRSSTRDWKDHKFKWRKDPQDK
Intracellular 2B4
(SEQ ID NO: 13)
tggaggagaaagaggaaggagaagcagtcagagaccagtcccaaggaatttttgacaatttacgaagatgtcaaggatctgaaaacc
aggagaaatcacgagcaggagcagacttttcctggaggggggagcaccatctactctatgatccagtcccagtcttctgctcccacgt
cacaagaacctgcatatacattatattcattaattcagccttccaggaagtctggatccaggaagaggaaccacagcccttccttcaatag
cactatctatgaagtgattggaaagagtcaacctaaagcccagaaccctgctcgattgagccgcaaagagctggagaactttgatgttt
attcc
(SEQ ID NO: 14)
WRRKRKEKQSETSPKEFLTIYEDVKDLKTRRNHEQEQTFPGGGSTIYSMIQSQSSAPT
SQEPAYTLYSLIQPSRKSGSRKRNHSPSFNSTIYEVIGKSQPKAQNPARLSRKELENFD
VYS
Intracellular CD3ζ
(SEQ ID NO: 15)
agagtgaagttcagcaggagcgcagacgcccccgcgtaccagcagggccagaaccagctctataacgagctcaatctaggacgaa
gagaggagtacgatgttttggacaagagacgtggccgggaccctgagatggggggaaagccgagaaggaagaaccctcaggaag
gcctgtacaatgaactgcagaaagataagatggcggaggcctacagtgagattgggatgaaaggcgagcgccggaggggcaagg
ggcacgatggcctttaccagggtctcagtacagccaccaaggacacctacgacgcccttcacatgcaggccctgcctcctcgc
(SEQ ID NO: 16)
RVKFSRSADAPAYQQGQNQLYNELNLGRREEYDVLDKRRGRDPEMGGKPRRKNPQ
EGLYNELQKDKMAEAYSEIGMKGERRRGKGHDGLYQGLSTATKDTYDALHMQAL
PPR
Wild-type IL-15
(SEQ ID NO: 17)
atgagaatttcgaaaccacatttgagaagtatttccatccagtgctacttgtgtttacttctaaacagtcattttctaactgaagctggcattca
tgtcttcattttgggctgtttcagtgcagggcttcctaaaacagaagccaactgggtgaatgtaataagtgatttgaaaaaaattgaagatc
ttattcaatctatgcatattgatgctactttatatacggaaagtgatgttcaccccagttgcaaagtaacagcaatgaagtgctttctcttgga
gttacaagttatttcacttgagtccggagatgcaagtattcatgatacagtagaaaatctgatcatcctagcaaacaacagtttgtcttctaa
tgggaatgtaacagaatctggatgcaaagaatgtgaggaactggaggaaaaaaatattaaagaatttttgcagagttttgtacatattgtc
caaatgttcatcaacacttct
(SEQ ID NO: 18)
MRISKPHLRSISIQCYLCLLLNSHFLTEAGIHVFILGCFSAGLPKTEANWVNVISDLKKI
EDLIQSMHIDATLYTESDVHPSCKVTAMKCFLLELQVISLESGDASIHDTVENLIILAN
NSLSSNGNVTESGCKECEELEEKNIKEFLQSFVHIVQMFINTS
Intracellular common gamma chain(γc)
(SEQ ID NO: 19)
gaacggacgatgccccgaattcccaccctgaagaacctagaggatcttgttactgaataccacgggaacttttcggcctggagtggtgt
gtctaagggactggctgagagtctgcagccagactacagtgaacgactctgcctcgtcagtgagattcccccaaaaggaggggccct
tggggaggggcctggggcctccccatgcaaccagcatagcccctactgggcccccccatgttacaccctaaagcctgaaacc
(SEQ ID NO: 20)
ERTMPRIPTLKNLEDLVTEYHGNFSAWSGVSKGLAESLQPDYSERLCLVSEIPPKGGA
LGEGPGASPCNQHSPYWAPPCYTLKPET
CD16A Transmembrane
(SEQ ID NO: 21)
gtctctttctgcttggtgatggtactcctttttgcagtggacacaggactatatttctctgtg
(SEQ ID NO: 22)
VSFCLVMVLLFAVDTGLYFSV
2B4 Transmembrane
(SEQ ID NO: 23)
tttttggtgatcatcgtgattctaagcgcactgttccttggcacccttgcctgcttctgtgtg
(SEQ ID NO: 24)
FLVIIVILSALFLGTLACFCV
NKG2D Transmembrane
(SEQ ID NO: 25)
ccattttttttctgctgcttcatcgctgtagccatgggaatccgtttcattattatggtagca
(SEQ ID NO: 26)
PFFFCCFIAVAMGIRFIIMVA
CD28 TM domain + CD16A + 2B4 + CD3z
(SEQ ID NO: 27)
actagtgccgtgcagggccccgaggagaccgtgacccaggactgcctgcagctgatcgccgacagcgagacccccaccatccag
aagggcagctacaccttcgtgccctggctgctgagcttcaagaggggcagcgccctggaggagaaggagaacaagatcctggtga
aggagaccggctacttcttcatctacggccaggtgctgtacaccgacaagacctacgccatgggccacctgatccagaggaagaag
gtgcacgtgttcggcgacgagctgagcctggtgaccctgttcaggtgcatccagaacatgcccgagaccctgcccaacaacagctgc
tacagcgccggcatcgccaagctggaggagggcgacgagctgcagctggccatccccagggagaacgcccagatcagcctggac
ggcgacgtgaccttcttcggcgccctgaagctgctgtctggaggcggctcggatcccgccgagcccaaatctcctgacaaaactcac
acatgcccaccgtgcccaaaagatcccaaattttgggtgctggtggtggttggtggagtcctggcttgctatagcttgctagtaacagtg
gcctttattattttctgggtgaagacaaacattcgaagctcaacaagagactggaaggaccataaatttaaatggagaaaggaccctcaa
gacaaatggaggagaaagaggaaggagaagcagtcagagaccagtcccaaggaatttttgacaatttacgaagatgtcaaggatctg
aaaaccaggagaaatcacgagcaggagcagacttttcctggaggggggagcaccatctactctatgatccagtcccagtcttctgctc
ccacgtcacaagaacctgcatatacattatattcattaattcagccttccaggaagtctggatccaggaagaggaaccacagcccttcctt
caatagcactatctatgaagtgattggaaagagtcaacctaaagcccagaaccctgctcgattgagccgcaaagagctggagaacttt
gatgtttattccagagtgaagttcagcaggagcgcagacgcccccgcgtaccagcagggccagaaccagctctataacgagctcaat
ctaggacgaagagaggagtacgatgttttggacaagagacgtggccgggaccctgagatggggggaaagccgagaaggaagaac
cctcaggaaggcctgtacaatgaactgcagaaagataagatggcggaggcctacagtgagattgggatgaaaggcgagcgccgga
ggggcaaggggcacgatggcctttaccagggtctcagtacagccaccaaggacacctacgacgcccttcacatgcaggccctgcct
cctcgctcgcgaggatctggagcaacaaacttctcactactcaaacaagcaggtgacgtggaggagaatcccgggccttctagacct
aggatgagaatttcgaaaccacatttgagaagtatttccatccagtgctacttgtgtttacttctaaacagtcattttctaactgaagctggca
ttcatgtcttcattttgggctgtttcagtgcagggcttcctaaaacagaagccaactgggtgaatgtaataagtgatttgaaaaaaattgaa
gatcttattcaatctatgcatattgatgctactttatatacggaaagtgatgttcaccccagttgcaaagtaacagcaatgaagtgctttctctt
ggagttacaagttatttcacttgagtccggagatgcaagtattcatgatacagtagaaaatctgatcatcctagcaaacaacagtttgtctt
ctaatgggaatgtaacagaatctggatgcaaagaatgtgaggaactggaggaaaaaaatattaaagaatttttgcagagttttgtacatat
tgtccaaatgttcatcaacacttcttgatacgta
CD28 TM domain + common gamma chain + 2B4 + CD3z
(SEQ ID NO: 28)
actagtgccgtgcagggccccgaggagaccgtgacccaggactgcctgcagctgatcgccgacagcgagacccccaccatccag
aagggcagctacaccttcgtgccctggctgctgagcttcaagaggggcagcgccctggaggagaaggagaacaagatcctggtga
aggagaccggctacttcttcatctacggccaggtgctgtacaccgacaagacctacgccatgggccacctgatccagaggaagaag
gtgcacgtgttcggcgacgagctgagcctggtgaccctgttcaggtgcatccagaacatgcccgagaccctgcccaacaacagctgc
tacagcgccggcatcgccaagctggaggagggcgacgagctgcagctggccatccccagggagaacgcccagatcagcctggac
ggcgacgtgaccttcttcggcgccctgaagctgctgtctggaggcggctcggatcccgccgagcccaaatctcctgacaaaactcac
acatgcccaccgtgcccaaaagatcccaaattttgggtgctggtggtggttggtggagtcctggcttgctatagcttgctagtaacagtg
gcctttattattttctgggtggaacggacgatgccccgaattcccaccctgaagaacctagaggatcttgttactgaataccacgggaact
tttcggcctggagtggtgtgtctaagggactggctgagagtctgcagccagactacagtgaacgactctgcctcgtcagtgagattccc
ccaaaaggaggggcccttggggaggggcctggggcctccccatgcaaccagcatagcccctactgggcccccccatgttacaccct
aaagcctgaaacctggaggagaaagaggaaggagaagcagtcagagaccagtcccaaggaatttttgacaatttacgaagatgtca
aggatctgaaaaccaggagaaatcacgagcaggagcagacttttcctggaggggggagcaccatctactctatgatccagtcccagt
cttctgctcccacgtcacaagaacctgcatatacattatattcattaattcagccttccaggaagtctggatccaggaagaggaaccaca
gcccttccttcaatagcactatctatgaagtgattggaaagagtcaacctaaagcccagaaccctgctcgattgagccgcaaagagctg
gagaactttgatgtttattccagagtgaagttcagcaggagcgcagacgcccccgcgtaccagcagggccagaaccagctctataac
gagctcaatctaggacgaagagaggagtacgatgttttggacaagagacgtggccgggaccctgagatggggggaaagccgagaa
ggaagaaccctcaggaaggcctgtacaatgaactgcagaaagataagatggcggaggcctacagtgagattgggatgaaaggcga
gcgccggaggggcaaggggcacgatggcctttaccagggtctcagtacagccaccaaggacacctacgacgcccttcacatgcag
gccctgcctcctcgctcgcgaggatctggagcaacaaacttctcactactcaaacaagcaggtgacgtggaggagaatcccgggcct
tctagacctaggatgagaatttcgaaaccacatttgagaagtatttccatccagtgctacttgtgtttacttctaaacagtcattttctaactga
agctggcattcatgtcttcattttgggctgtttcagtgcagggcttcctaaaacagaagccaactgggtgaatgtaataagtgatttgaaaa
aaattgaagatcttattcaatctatgcatattgatgctactttatatacggaaagtgatgttcaccccagttgcaaagtaacagcaatgaagt
gctttctcttggagttacaagttatttcacttgagtccggagatgcaagtattcatgatacagtagaaaatctgatcatcctagcaaacaaca
gtttgtcttctaatgggaatgtaacagaatctggatgcaaagaatgtgaggaactggaggaaaaaaatattaaagaatttttgcagagtttt
gtacatattgtccaaatgttcatcaacacttcttgatacgta
CD16A TM domain + CD16A + 2B4 + CD3z
(SEQ ID NO: 29)
actagtgccgtgcagggccccgaggagaccgtgacccaggactgcctgcagctgatcgccgacagcgagacccccaccatccag
aagggcagctacaccttcgtgccctggctgctgagcttcaagaggggcagcgccctggaggagaaggagaacaagatcctggtga
aggagaccggctacttcttcatctacggccaggtgctgtacaccgacaagacctacgccatgggccacctgatccagaggaagaag
gtgcacgtgttcggcgacgagctgagcctggtgaccctgttcaggtgcatccagaacatgcccgagaccctgcccaacaacagctgc
tacagcgccggcatcgccaagctggaggagggcgacgagctgcagctggccatccccagggagaacgcccagatcagcctggac
ggcgacgtgaccttcttcggcgccctgaagctgctgtctggaggggctcggatcccgccgagcccaaatctcctgacaaaactcac
acatgcccaccgtgcccaaaagatcccaaagtctctttctgcttggtgatggtactcctttttgcagtggacacaggactatatttctctgtg
aagacaaacattcgaagctcaacaagagactggaaggaccataaatttaaatggagaaaggaccctcaagacaaatggaggagaaa
gaggaaggagaagcagtcagagaccagtcccaaggaatttttgacaatttacgaagatgtcaaggatctgaaaaccaggagaaatca
cgagcaggagcagacttttcctggaggggggagcaccatctactctatgatccagtcccagtcttctgctcccacgtcacaagaacctg
catatacattatattcattaattcagccttccaggaagtctggatccaggaagaggaaccacagcccttccttcaatagcactatctatgaa
gtgattggaaagagtcaacctaaagcccagaaccctgctcgattgagccgcaaagagctggagaactttgatgtttattccagagtgaa
gttcagcaggagcgcagacgcccccgcgtaccagcagggccagaaccagctctataacgagctcaatctaggacgaagagagga
gtacgatgttttggacaagagacgtggccgggaccctgagatggggggaaagccgagaaggaagaaccctcaggaaggcctgtac
aatgaactgcagaaagataagatggcggaggcctacagtgagattgggatgaaaggcgagcgccggaggggcaaggggcacgat
ggcctttaccagggtctcagtacagccaccaaggacacctacgacgcccttcacatgcaggccctgcctcctcgctcgcgaggatctg
gagcaacaaacttctcactactcaaacaagcaggtgacgtggaggagaatcccgggccttctagacctaggatgagaatttcgaaacc
acatttgagaagtatttccatccagtgctacttgtgtttacttctaaacagtcattttctaactgaagctggcattcatgtcttcattttgggctgt
ttcagtgcagggcttcctaaaacagaagccaactgggtgaatgtaataagtgatttgaaaaaaattgaagatcttattcaatctatgcatat
tgatgctactttatatacggaaagtgatgttcaccccagttgcaaagtaacagcaatgaagtgctttctcttggagttacaagttatttcactt
gagtccggagatgcaagtattcatgatacagtagaaaatctgatcatcctagcaaacaacagtttgtcttctaatgggaatgtaacagaat
ctggatgcaaagaatgtgaggaactggaggaaaaaaatattaaagaatttttgcagagttttgtacatattgtccaaatgttcatcaacact
tcttgatacgta
2B4 TM domain+ CD16A + 2B4 + CD3z
(SEQ ID NO: 30)
actagtgccgtgcagggccccgaggagaccgtgacccaggactgcctgcagctgatcgccgacagcgagacccccaccatccag
aagggcagctacaccttcgtgccctggctgctgagcttcaagaggggcagcgccctggaggagaaggagaacaagatcctggtga
aggagaccggctacttcttcatctacggccaggtgctgtacaccgacaagacctacgccatgggccacctgatccagaggaagaag
gtgcacgtgttcggcgacgagctgagcctggtgaccctgttcaggtgcatccagaacatgcccgagaccctgcccaacaacagctgc
tacagcgccggcatcgccaagctggaggagggcgacgagctgcagctggccatccccagggagaacgcccagatcagcctggac
ggcgacgtgaccttcttcggcgccctgaagctgctgtctggaggcggctcggatcccgccgagcccaaatctcctgacaaaactcac
acatgcccaccgtgcccaaaagatcccaaatttttggtgatcatcgtgattctaagcgcactgttccttggcacccttgcctgcttctgtgt
gaagacaaacattcgaagctcaacaagagactggaaggaccataaatttaaatggagaaaggaccctcaagacaaatggaggagaa
agaggaaggagaagcagtcagagaccagtcccaaggaatttttgacaatttacgaagatgtcaaggatctgaaaaccaggagaaatc
acgagcaggagcagacttttcctggaggggggagcaccatctactctatgatccagtcccagtcttctgctcccacgtcacaagaacct
gcatatacattatattcattaattcagccttccaggaagtctggatccaggaagaggaaccacagcccttccttcaatagcactatctatga
agtgattggaaagagtcaacctaaagcccagaaccctgctcgattgagccgcaaagagctggagaactttgatgtttattccagagtga
agttcagcaggagcgcagacgcccccgcgtaccagcagggccagaaccagctctataacgagctcaatctaggacgaagagagg
agtacgatgttttggacaagagacgtggccgggaccctgagatggggggaaagccgagaaggaagaaccctcaggaaggcctgta
caatgaactgcagaaagataagatggcggaggcctacagtgagattgggatgaaaggcgagcgccggaggggcaaggggcacg
atggcctttaccagggtctcagtacagccaccaaggacacctacgacgcccttcacatgcaggccctgcctcctcgctcgcgaggatc
tggagcaacaaacttctcactactcaaacaagcaggtgacgtggaggagaatcccgggccttctagacctaggatgagaatttcgaaa
ccacatttgagaagtatttccatccagtgctacttgtgtttacttctaaacagtcattttctaactgaagctggcattcatgtcttcattttgggc
tgtttcagtgcagggcttcctaaaacagaagccaactgggtgaatgtaataagtgatttgaaaaaaattgaagatcttattcaatctatgca
tattgatgctactttatatacggaaagtgatgttcaccccagttgcaaagtaacagcaatgaagtgctttctcttggagttacaagttatttca
cttgagtccggagatgcaagtattcatgatacagtagaaaatctgatcatcctagcaaacaacagtttgtcttctaatgggaatgtaacag
aatctggatgcaaagaatgtgaggaactggaggaaaaaaatattaaagaatttttgcagagttttgtacatattgtccaaatgttcatcaac
acttcttgatacgta
NKG2D TM domain + CD16A + 2B4 + CD3z
(SEQ ID NO: 31)
actagtgccgtgcagggccccgaggagaccgtgacccaggactgcctgcagctgatcgccgacagcgagacccccaccatccag
aagggcagctacaccttcgtgccctggctgctgagcttcaagaggggcagcgccctggaggagaaggagaacaagatcctggtga
aggagaccggctacttcttcatctacggccaggtgctgtacaccgacaagacctacgccatgggccacctgatccagaggaagaag
gtgcacgtgttcggcgacgagctgagcctggtgaccctgttcaggtgcatccagaacatgcccgagaccctgcccaacaacagctgc
tacagcgccggcatcgccaagctggaggagggcgacgagctgcagctggccatccccagggagaacgcccagatcagcctggac
ggcgacgtgaccttcttcggcgccctgaagctgctgtctggaggcggctcggatcccgccgagcccaaatctcctgacaaaactcac
acatgcccaccgtgcccaaaagatcccaaaccattttttttctgctgcttcatcgctgtagccatgggaatccgtttcattattatggtagca
aagacaaacattcgaagctcaacaagagactggaaggaccataaatttaaatggagaaaggaccctcaagacaaatggaggagaaa
gaggaaggagaagcagtcagagaccagtcccaaggaatttttgacaatttacgaagatgtcaaggatctgaaaaccaggagaaatca
cgagcaggagcagacttttcctggaggggggagcaccatctactctatgatccagtcccagtcttctgctcccacgtcacaagaacctg
catatacattatattcattaattcagccttccaggaagtctggatccaggaagaggaaccacagcccttccttcaatagcactatctatgaa
gtgattggaaagagtcaacctaaagcccagaaccctgctcgattgagccgcaaagagctggagaactttgatgtttattccagagtgaa
gttcagcaggagcgcagacgcccccgcgtaccagcagggccagaaccagctctataacgagctcaatctaggacgaagagagga
gtacgatgttttggacaagagacgtggccgggaccctgagatggggggaaagccgagaaggaagaaccctcaggaaggcctgtac
aatgaactgcagaaagataagatggcggaggcctacagtgagattgggatgaaaggcgagcgccggaggggcaaggggcacgat
ggcctttaccagggtctcagtacagccaccaaggacacctacgacgcccttcacatgcaggccctgcctcctcgctcgcgaggatctg
gagcaacaaacttctcactactcaaacaagcaggtgacgtggaggagaatcccgggccttctagacctaggatgagaatttcgaaacc
acatttgagaagtatttccatccagtgctacttgtgtttacttctaaacagtcattttctaactgaagctggcattcatgtcttcattttgggctgt
ttcagtgcagggcttcctaaaacagaagccaactgggtgaatgtaataagtgatttgaaaaaaattgaagatcttattcaatctatgcatat
tgatgctactttatatacggaaagtgatgttcaccccagttgcaaagtaacagcaatgaagtgctttctcttggagttacaagttatttcactt
gagtccggagatgcaagtattcatgatacagtagaaaatctgatcatcctagcaaacaacagtttgtcttctaatgggaatgtaacagaat
ctggatgcaaagaatgtgaggaactggaggaaaaaaatattaaagaatttttgcagagttttgtacatattgtccaaatgttcatcaacact
tcttgatacgta
CD28 intracellular domain
(SEQ ID NO: 32)
aggagtaagaggagcaggctcctgcacagtgactacatgaacatgactccccgccgccccgggcccacccgcaagcattaccagc
cctatgccccaccacgcgacttcgcagcctatcgctcc
(SEQ ID NO: 33)
RSKRSRLLHSDYMNMTPRRPGPTRKHYQPYAPPRDFAAYRS
41BB intracellular domain
(SEQ ID NO: 34)
aaacggggcagaaagaaactcctgtatatattcaaacaaccatttatgagaccagtacaaactactcaagaggaagatggctgtagctg
ccgatttccagaagaagaagaaggaggatgtgaactg
(SEQ ID NO: 35)
KRGRKKLLYIFKQPFMRPVQTTQEEDGCSCRFPEEEEGGCEL
DAP10 intracellular domain
(SEQ ID NO: 36)
ctgtgcgcacgcccacgccgcagccccgcccaagaagatggcaaagtctacatcaacatgccaggcaggggc
(SEQ ID NO: 37)
LCARPRRSPAQEDGKVYINMPGRG
DAP12 intracellular domain
(SEQ ID NO: 38)
tacttcctgggccggctggtccctcgggggcgaggggctgcggaggcagcgacccggaaacagcgtatcactgagaccgagtcgc
cttatcaggagctccagggtcagaggtcggatgtctacagcgacctcaacacacagaggccgtattacaaa
(SEQ ID NO: 39)
YFLGRLVPRGRGAAEAATRKQRITETESPYQELQGQRSDVYSDLNTQRPYYK
CD28 TM domain + 2B4 + DAP10 + DAP12 + CD3z
(SEQ ID NO: 40)
actagtgccgtgcagggccccgaggagaccgtgacccaggactgcctgcagctgatcgccgacagcgagacccccaccatccag
aagggcagctacaccttcgtgccctggctgctgagcttcaagaggggcagcgccctggaggagaaggagaacaagatcctggtga
aggagaccggctacttcttcatctacggccaggtgctgtacaccgacaagacctacgccatgggccacctgatccagaggaagaag
gtgcacgtgttcggcgacgagctgagcctggtgaccctgttcaggtgcatccagaacatgcccgagaccctgcccaacaacagctgc
tacagcgccggcatcgccaagctggaggagggcgacgagctgcagctggccatccccagggagaacgcccagatcagcctggac
ggcgacgtgaccttcttcggcgccctgaagctgctgtctggaggcggctcggatcccgccgagcccaaatctcctgacaaaactcac
acatgcccaccgtgcccaaaagatcccaaattttgggtgctggtggtggttggtggagtcctggcttgctatagcttgctagtaacagtg
gcctttattattttctgggtgtggaggagaaagaggaaggagaagcagtcagagaccagtcccaaggaatttttgacaatttacgaagat
gtcaaggatctgaaaaccaggagaaatcacgagcaggagcagacttttcctggaggggggagcaccatctactctatgatccagtcc
cagtcttctgctcccacgtcacaagaacctgcatatacattatattcattaattcagccttccaggaagtctggatccaggaagaggaacc
acagcccttccttcaatagcactatctatgaagtgattggaaagagtcaacctaaagcccagaaccctgctcgattgagccgcaaagag
ctggagaactttgatgtttattccctgtgcgcacgcccacgccgcagccccgcccaagaagatggcaaagtctacatcaacatgccag
gcaggggctacttcctgggccggctggtccctcgggggcgaggggctgcggaggcagcgacccggaaacagcgtatcactgaga
ccgagtcgccttatcaggagctccagggtcagaggtcggatgtctacagcgacctcaacacacagaggccgtattacaaaagagtga
agttcagcaggagcgcagacgcccccgcgtaccagcagggccagaaccagctctataacgagctcaatctaggacgaagagagg
agtacgatgttttggacaagagacgtggccgggaccctgagatggggggaaagccgagaaggaagaaccctcaggaaggcctgta
caatgaactgcagaaagataagatggcggaggcctacagtgagattgggatgaaaggcgagcgccggaggggcaaggggcacg
atggcctttaccagggtctcagtacagccaccaaggacacctacgacgcccttcacatgcaggccctgcctcctcgccatatg
CD28 TM domain + CD28 +2B4 + CD3z
(SEQ ID NO: 41)
actagtgccgtgcagggccccgaggagaccgtgacccaggactgcctgcagctgatcgccgacagcgagacccccaccatccag
aagggcagctacaccttcgtgccctggctgctgagcttcaagaggggcagcgccctggaggagaaggagaacaagatcctggtga
aggagaccggctacttcttcatctacggccaggtgctgtacaccgacaagacctacgccatgggccacctgatccagaggaagaag
gtgcacgtgttcggcgacgagctgagcctggtgaccctgttcaggtgcatccagaacatgcccgagaccctgcccaacaacagctgc
tacagcgccggcatcgccaagctggaggagggcgacgagctgcagctggccatccccagggagaacgcccagatcagcctggac
ggcgacgtgaccttcttcggcgccctgaagctgctgtctggaggcggctcggatcccgccgagcccaaatctcctgacaaaactcac
acatgcccaccgtgcccaaaagatcccaaattttgggtgctggtggtggttggtggagtcctggcttgctatagcttgctagtaacagtg
gcctttattattttctgggtgaggagtaagaggagcaggctcctgcacagtgactacatgaacatgactccccgccgccccgggccca
cccgcaagcattaccagccctatgccccaccacgcgacttcgcagcctatcgctcctggaggagaaagaggaaggagaagcagtca
gagaccagtcccaaggaatttttgacaatttacgaagatgtcaaggatctgaaaaccaggagaaatcacgagcaggagcagacttttc
ctggaggggggagcaccatctactctatgatccagtcccagtcttctgctcccacgtcacaagaacctgcatalacattatattcattaatt
cagccttccaggaagtctggatccaggaagaggaaccacagcccttccttcaatagcactatctatgaagtgattggaaagagtcaac
ctaaagcccagaaccctgctcgattgagccgcaaagagctggagaactttgatgtttattccagagtgaagttcagcaggagcgcaga
cgcccccgcgtaccagcagggccagaaccagctctataacgagctcaatctaggacgaagagaggagtacgatgttttggacaaga
gacgtggccgggaccctgagatggggggaaagccgagaaggaagaaccctcaggaaggcctgtacaatgaactgcagaaagata
agatggcggaggcctacagtgagattgggatgaaaggcgagcgccggaggggcaaggggcacgatggcctttaccagggtctca
gtacagccaccaaggacacctacgacgcccttcacatgcaggccctgcctcctcgccatatg
CD28 TM domain + 2B4 + 41BB + CD3z
(SEQ ID NO: 42)
actagtgccgtgcagggccccgaggagaccgtgacccaggactgcctgcagctgatcgccgacagcgagacccccaccatccag
aagggcagctacaccttcgtgccctggctgctgagcttcaagaggggcagcgccctggaggagaaggagaacaagatcctggtga
aggagaccggctacttcttcatctacggccaggtgctgtacaccgacaagacctacgccatgggccacctgatccagaggaagaag
gtgcacgtgttcggcgacgagctgagcctggtgaccctgttcaggtgcatccagaacatgcccgagaccctgcccaacaacagctgc
tacagcgccggcatcgccaagctggaggagggcgacgagctgcagctggccatccccagggagaacgcccagatcagcctggac
ggcgacgtgaccttcttcggcgccctgaagctgctgtctggaggcggctcggatcccgccgagcccaaatctcctgacaaaactcac
acatgcccaccgtgcccaaaagatcccaaattttgggtgctggtggtggttggtggagtcctggcttgctatagcttgctagtaacagtg
gcctttattattttctgggtgtggaggagaaagaggaaggagaagcagtcagagaccagtcccaaggaatttttgacaatttacgaagat
gtcaaggatctgaaaaccaggagaaatcacgagcaggagcagacttttcctggaggggggagcaccatctactctatgatccagtcc
cagtcttctgctcccacgtcacaagaacctgcatatacattatattcattaattcagccttccaggaagtctggatccaggaagaggaacc
acagcccttccttcaatagcactatctatgaagtgattggaaagagtcaacctaaagcccagaaccctgctcgattgagccgcaaagag
ctggagaactttgatgtttattccaaacggggcagaaagaaactcctgtatatattcaaacaaccatttatgagaccagtacaaactactc
aagaggaagatggctgtagctgccgatttccagaagaagaagaaggaggatgtgaactgagagtgaagttcagcaggagcgcaga
cgcccccgcgtaccagcagggccagaaccagctctataacgagctcaatctaggacgaagagaggagtacgatgttttggacaaga
gacgtggccgggaccctgagatggggggaaagccgagaaggaagaaccctcaggaaggcctgtacaatgaactgcagaaagata
agatggcggaggcctacagtgagattgggatgaaaggcgagcgccggaggggcaaggggcacgatggcctttaccagggtctca
gtacagccaccaaggacacctacgacgcccttcacatgcaggccctgcctcctcgccatatg
TIGIT extracellular domain
(SEQ ID NO: 43)
Atgcgctggtgtctcctcctgatctgggcccaggggctgaggcaggctcccctcgcctcaggaatgatgacaggcacaatagaaac
aacggggaacatttctgcagagaaaggtggctctatcatcttacaatgtcacctctcctccaccacggcacaagtgacccaggtcaact
gggagcagcaggaccagcttctggccatttgtaatgctgacttggggggcacatctccccatccttcaaggatcgagtggccccagg
tcccggcctgggcctcaccctccagtcgctgaccgtgaacgatacaggggagtacttctgcatctatcacacctaccctgatgggacgt
acactgggagaatcttcctggaggtcctagaaagctcagtggctgagcacggtgccaggttccagattcca
(SEQ ID NO: 44)
MRWCLLLIWAQGLRQAPLASGMMTGTIETTGNISAEKGGSIILQCHLSSTTAQVTQV
NWEQQDQLLAICNADLGWHISPSFKDRVAPGPGLGLTLQSLTVNDTGEYFCIYHTYP
DGTYTGRIFLEVLESSVAEHGARFQIP
PD-1 extracellular domain
(SEQ ID NO: 45)
Atgcagatcccacaggcgccctggccagtcgtctgggcggtgctacaactgggctggcggccaggatggttcttagactccccaga
caggccctggaacccccccaccttctccccagccctgctcgtggtgaccgaaggggacaacgccaccttcacctgcagcttctccaa
cacatcggagagcttcgtgctaaactggtaccgcatgagccccagcaaccagacggacaagctggccgccttccccgaggaccgc
agccagcccggccaggactgccgcttccgtgtcacacaactgcccaacgggcgtgacttccacatgagcgtggtcagggcccggc
gcaatgacagcggcacctacctctgtggggccatctccctggcccccaaggcgcagatcaaagagagcctgcgggcagagctcag
ggtgacagagagaagggcagaagtgcccacagcccaccccagcccctcacccaggccagccggccagttccaaaccctggtg
(SEQ ID NO: 46)
MQIPQAPWPVVWAVLQLGWRPGWFLDSPDRPWNPPTFSPALLVVTEGDNATFTCSF
SNTSESFVLNWYRMSPSNQTDKLAAFPEDRSQPGQDCRFRVTQLPNGRDFHMSVVR
ARRNDSGTYLCGAISLAPKAQIKESLRAELRVTERRAEVPTAHPSPSPRPAGQFQTLV
IL-21R transmembrane and intracellular domains
(SEQ ID NO: 47)
ggctggaaccctcacctgctgcttctcctcctgcttgtcatagtcttcattcctgccttctggagcctgaagacccatccattgtggaggct
atggaagaagatatgggccgtccccagccctgagcggttcttcatgcccctgtacaagggctgcagcggagacttcaagaaatgggt
gggtgcacccttcactggctccagcctggagctgggaccctggagcccagaggtgccctccaccctggaggtgtacagctgccacc
caccacggagcccggccaagaggctgcagctcacggagctacaagaaccagcagagctggtggagtctgacggtgtgcccaagc
ccagcttctggccgacagcccagaactcggggggctcagcttacagtgaggagagggatcggccatacggcctggtgtccattgac
acagtgactgtgctagatgcagaggggccatgcacctggccctgcagtgtgaggatgacggctacccagccctggacctggatgct
ggcctggagcccagcccaggcctagaggacccactcttggatgcagggaccacagtcctgtcctgtggctgtgtctcagctggcagc
cctgggctaggagggcccctgggaagcctcctggacagactaaagccaccccttgcagatggggaggactgggctgggggactgc
cctggggtggccggtcacctggaggggtctcagagagtgaggcgggctcacccctggccggcctggatatggacacgtttgacagt
ggctttgtgggctctgactgcagcagccctgtggagtgtgacttcaccagccccggggacgaaggacccccccggagctacctccg
ccagtgggtggtcattcctccgccactttcgagccctggaccccaggccagc
(SEQ ID NO: 48)
GWNPHLLLLLLLVIVFIPAFWSLKTHPLWRLWKKIWAVPSPERFFMPLYKGCSGDFK
KWVGAPFTGSSLELGPWSPEVPSTLEVYSCHPPRSPAKRLQLTELQEPAELVESDGVP
KPSFWPTAQNSGGSAYSEERDRPYGLVSIDTVTVLDAEGPCTWPCSCEDDGYPALDL
DAGLEPSPGLEDPLLDAGTTVLSCGCVSAGSPGLGGPLGSLLDRLKPPLADGEDWAG
GLPWGGRSPGGVSESEAGSPLAGLDMDTFDSGFVGSDCSSPVECDFTSPGDEGPPRS
YLRQWVVIPPPLSSPGPQAS
TIGIT-IL-21-R
(SEQ ID NO: 49)
actagtgccaccatgcgctggtgtctcctcctgatctgggcccaggggctgaggcaggctcccctcgcctcaggaatgatgacaggc
acaatagaaacaacggggaacatttctgcagagaaaggtggctctatcatcttacaatgtcacctctcctccaccacggcacaagtgac
ccaggtcaactgggagcagcaggaccagcttctggccatttgtaatgctgacttggggggcacatctccccatccttcaaggatcgag
tggccccaggtcccggcctgggcctcaccctccagtcgctgaccgtgaacgatacaggggagtacttctgcatctatcacacctaccc
tgatgggacgtacactgggagaatcttcctggaggtcctagaaagctcagtggctgagcacggtgccaggttccagattccaggctgg
aaccctcacctgctgcttctcctcctgcttgtcatagtcttcattcctgccttctggagcctgaagacccatccattgtggaggctatggaa
gaagatatgggccgtccccagccctgagcggttcttcatgcccctgtacaagggctgcagcggagacttcaagaaatggggggtgc
acccttactggctccagcctggagctgggaccctggagcccagaggtgccctccaccctggaggtgtacagctgccacccaccac
ggagcccggccaagaggctgcagctcacggagctacaagaaccagcagagctggtggagtctgacggtgtgcccaagcccagctt
ctggccgacagcccagaactcggggggctcagcttacagtgaggagagggatcggccatacggcctggtgtccattgacacagtga
ctgtgctagatgcagaggggccatgcacctggccctgcagctgtgaggatgacggctacccagccctggacctggatgctggcctg
gagcccagcccaggcctagaggacccactcttggatgcagggaccacagtcctgtcctgtggctgtgtctcagctggcagccctggg
ctaggagggcccctgggaagcctcctggacagactaaagccaccccttgcagatggggaggactgggctgggggactgccctggg
gtggccggtcacctggaggggtctcagagagtgaggcgggctcacccctggccggcctggatatggacacgtttgacagtggctttg
tgggctctgactgcagcagccctgtggagtgtgacttcaccagccccggggacgaaggacccccccggagctacctccgccagtgg
gtggtcattcctccgccactttcgagccctggaccccaggccagccatatg
PD-1-IL-21-R
(SEQ ID NO: 50)
actagtgccaccatgcagatcccacaggcgccctggccagtcgtctgggcggtgctacaactgggctggcggccaggatggttctta
gactccccagacaggccctggaacccccccaccttctccccagccctgctcgtggtgaccgaaggggacaacgccaccttcacctg
cagcttctccaacacatcggagagcttcgtgctaaactggtaccgcatgagccccagcaaccagacggacaagctggccgccttccc
cgaggaccgcagccagcccggccaggactgccgcttccgtgtcacacaactgcccaacgggcgtgacttccacatgagcgtggtca
gggcccggcgcaatgacagcggcacctacctctgtggggccatctccctggcccccaaggcgcagatcaaagagagcctgcggg
cagagctcagggtgacagagagaagggcagaagtgcccacagcccaccccagcccctcacccaggccagccggccagttccaaa
ccctggtgggctggaaccctcacctgctgcttctcctcctgcttgtcatagtcttcattcctgccttctggagcctgaagacccatccattgt
ggaggctatggaagaagatatgggccgtccccagccctgagcggttcttcatgcccctgtacaagggctgcagcggagacttcaaga
aatggggggtgcacccttcactggctccagcctggagctgggaccctggagcccagaggtgccctccaccctggaggtgtacagct
gccacccaccacggagcccggccaagaggctgcagctcacggagctacaagaaccagcagagctggtggagtctgacggtgtgc
ccaagcccagcttctggccgacagcccagaactcggggggctcagcttacagtgaggagagggatcggccatacggcctggtgtc
cattgacacagtgactgtgctagatgcagaggggccatgcacctggccctgcagctgtgaggatgacggctacccagccctggacct
ggatgctggcctggagcccagcccaggcctagaggacccactcttggatgcagggaccacagtcctgtcctgtggctgtgtctcagc
tggcagccctgggctaggagggcccctgggaagcctcctggacagactaaagccaccccttgcagatggggaggactgggctggg
ggactgccctggggggccggtcacctggaggggtctcagagagtgaggcgggctcacccctggccggcctggatatggacacgt
ttgacagtggctttgtgggctctgactgcagcagccctgtggagtgtgacttcaccagccccggggacgaaggacccccccggagct
acctccgccagtgggggtcattcctccgccactttcgagccctggaccccaggccagccatatg

From the above description of the invention, those skilled in the art will perceive improvements, changes and modifications. Such improvements, changes and modifications within the skill of the art are intended to be covered by the appended claims. All references, publications, and patents cited in the present application are herein incorporated by reference in their entirety.

1: A chimeric antigen receptor (CAR) comprising: an extracellular antigen binding domain, a transmembrane domain, and at least one an intracellular signaling domain, wherein the intracellular signaling domain includes a CD16A intracellular signaling domain, γc intracellular signaling domain, 2B4 intracellular signaling domain, CD28 intracellular signaling domain, 41 BB intracellular signaling domain, DAP10 intracellular signaling domain, DAP12 intracellular signaling domain, and/or combinations thereof.

2: The CAR of claim 1, wherein the transmembrane is selected from a CD28 transmembrane domain, a CD16a transmembrane domain, a 2B4 transmembrane domain, or a NKG2D transmembrane domain.

3: The CAR of claim 2, wherein the CD28 transmembrane comprises an amino acid sequence at least about 90% identical to SEQ ID NO: 10;
the CD16a transmembrane comprises an amino acid sequence at least about 90% identical to SEQ ID NO: 22;
the 2B4 transmembrane comprises an amino acid sequence at least about 90%, identical to SEQ ID NO: 24; or
the NKG2D transmembrane comprises an amino acid sequence at least about 90% identical to SEQ ID NO: 26.

4-6. (canceled)

7: The CAR of claim 1, wherein the CD16a intracellular signaling domain comprises an amino acid sequence at least about 90% identical to SEQ ID NO: 12; or
the γc intracellular signaling domain comprises an amino acid sequence at least about 90% identical to SEQ ID NO: 20.

8. (canceled)

9: The CAR of claim 1, further comprising the 2B4 intracellular signaling domain and a CD3ζ intracellular domain.

10: The CAR of claim 9, wherein the 2B4 intracellular signaling domain comprises an amino acid sequence at least about 90% identical to SEQ ID NO: 14; and the CD3ζ intracellular domain includes an amino acid sequence at least about 90% identical to SEQ ID NO: 16.

11. (canceled)

12: The CAR of claim 1, further comprising at least one of an extracellular spacer or hinge domain.

13: The CAR of claim 12, wherein the hinge domain is an IgG1 hinge domain.

14: The CAR of claim 12, wherein the spacer comprises an amino acid sequence at least about 90% identical to SEQ ID NO: 6; or
the hinge domain comprises an amino acid sequence at least about 90% identical to SEQ ID NO: 8.

15. (canceled)

16: The CAR of claim 1, further comprising a signal peptide.

17: The CAR of claim 9, comprising the extracellular antigen binding domain, a spacer, an IgG1 hinge domain, the transmembrane domain, the CD16a intracellular signaling domain, the 2B4 intracellular signaling domain, and the CD3ζ intracellular domain.

18: The CAR of claim 9, comprising the extracellular antigen binding domain, a spacer, an IgG1 hinge domain, the transmembrane domain, γc intracellular signaling domain, the 2B4 intracellular signaling domain, and the CD3ζ intracellular domain.

19: The CAR of claim 9, comprising the extracellular antigen binding domain, a spacer, an IgG1 hinge domain, the transmembrane domain, the CD28 intracellular signaling domain, the 2B4 intracellular signaling domain, and the CD3ζ intracellular domain.

20: The CAR of claim 1, comprising the extracellular antigen binding domain, a spacer, an IgG1 hinge domain, the transmembrane domain, the 2B4 intracellular signaling domain, the 41 BB intracellular signaling domain, and a CD3ζ intracellular domain.

21: The CAR of claim 1, comprising the extracellular antigen binding domain, a spacer, an IgG1 hinge domain, the transmembrane domain, the 2B4 intracellular signaling domain, the DAP10 intracellular signaling domain, the DAP12 intracellular signaling domain, and a CD3ζ intracellular signaling domain.

22: The CAR of claim 1, wherein the extracellular antigen binding domain comprises a polypeptide that binds to a receptor of BAFF.

23: The CAR of claim 22, wherein the receptor of BAFF is selected from the group consisting of B-cell maturation antigen, transmembrane activator and CAML interactor, and BAFF receptor.

24: The CAR of claim 1, wherein the extracellular antigen binding domain comprises a BAFF ligand.

25: The CAR of claim 24, wherein the BAFF ligand comprises an amino acid sequence at least about 90% identical to SEQ ID NO: 4.

26-45. (canceled)

46: A natural killer cell comprising the CAR of claim 1.

47-61. (canceled)
